(19) United States
(12) Patent Application Publication
(10) Pub. No.: US 2024/0335239 A1
(43) Pub. Date: Oct. 10, 2024

(54) SURGICAL PATIENT AND/OR EQUIPMENT ALIGNMENT SYSTEMS AND METHODS

(71) Applicant: WTC Holdings llc, Shreveport, LA (US)

(72) Inventor: Wyche T Coleman, III, Shreveport, LA (US)

(73) Assignee: WTC Holdings llc, Shreveport, LA (US)

(21) Appl. No.: 18/629,426

(22) Filed: Apr. 8, 2024

Related U.S. Application Data
(60) Provisional application No. 63/457,429, filed on Apr. 6, 2023.
(60) Provisional application No. 63/534,663, filed on Aug. 25, 2023.

(51) Int. Cl. A61B 34/20; A61B 34/00; A61B 90/00

(52) U.S. Cl. CPC A61B 34/20; A61B 34/25; A61B 90/39; A61B 2090/373

(57) ABSTRACT

Surgical patient and/or equipment alignment systems suitable for establishing as an anchor point and/or facilitating optimal positioning of a patient and/or at least one surgical equipment unit for a surgical procedure in an operating room may include at least one of the following: a horizontal patient operating position corresponding to an optimal position of a surgical patient table configured to support the patient in the surgical procedure and at least one horizontal equipment operating position corresponding to an optimal position of the surgical equipment unit, respectively, in the surgical procedure. Surgical patent and/or equipment alignment methods and emitter positioning assemblies are also disclosed.

### CROSS-REFERENCE TO RELATED APPLICATIONS

This application claims the benefit of U.S. provisional application No. 63/457,429, filed Apr. 6, 2023, and entitled SURGICAL PATIENT ALIGNMENT SYSTEM AND METHOD, and U.S. provisional application No. 63/534,663, filed Aug. 25, 2023, and entitled SURGICAL PATIENT AND/OR EQUIPMENT ALINGMENT SYSTEMS AND METHODS, each of which provisional applications is hereby incorporated by reference herein in its entirety for all purposes.

### FIELD

Illustrative embodiments of the disclosure generally relate to positioning of patients and/or surgical equipment in an operating room for surgical procedures. More particularly, illustrative embodiments of the disclosure relate to surgical patient and/or equipment alignment systems and methods which utilize light beams to facilitate quick and precise positioning of a patient and/or surgical equipment for a surgical procedure.

### BACKGROUND

The background description provided herein is solely for the purpose of generally presenting the context of the illustrative embodiments of the disclosure. Aspects of the background description are neither expressly nor impliedly admitted as prior art against the claimed subject matter.

Surgical procedures typically require the use of specialized equipment. The surgical patient table which supports the patient and the surgical equipment in the operating room typically must be properly positioned with respect to the surgeon to optimize the ability of the surgeon to implement the procedure precisely and efficiently. Between surgeries, it may be necessary to clean the floor, walls, and other surfaces of the operating room. This may require that the surgical patient table and the equipment be moved from their operating positions to another position in the operating room or from the operating room completely to clean those areas of the operating room which are occupied by the equipment during surgery. After cleaning is completed, the surgical patient table and equipment must be moved back into their proper operating positions for implementation of the next surgical procedure. The operating positions of the surgical patient table and the equipment, however, are typically estimated by operating room personnel prior to the next procedure. This method, however, may be time-consuming since much guesswork may be required and an inordinate length of time may be expended moving the surgical patient table and the equipment back into the operating positions. Consequently, operating time may be lost, particularly under conditions of high case load when a large number of procedures need to be completed within a limited time period.

Accordingly, surgical patient alignment systems and methods which utilize light beams to facilitate quick and accurate positioning of a patient and/or surgical equipment for a surgical procedure may be desirable.

### SUMMARY

Illustrative embodiments of the disclosure are generally directed to surgical patient and/or equipment alignment systems for optimal positioning of a patient and/or at least one surgical equipment unit for a surgical procedure in an operating room. An illustrative embodiment of the system may include at least one of the following: a horizontal patient operating position corresponding to an optimal position of a surgical patient table configured to support the patient in the surgical procedure and a horizontal patient positioning light emitter directly above the horizontal patient operating position, the horizontal patient positioning light emitter configured to emit a downward light beam to impinge at a selected point on the patient when the surgical patient table is deployed in place at the horizontal patient operating position; and at least one horizontal equipment operating position corresponding to an optimal position of the at least one surgical equipment unit, respectively, in the surgical procedure, at least one horizontal equipment positioning light emitter configured for placement on the at least one surgical equipment unit, respectively, and at least one elevated light marker directly above the at least one horizontal equipment operating position, respectively, the at least one horizontal equipment positioning light emitter configured to emit at least one upward light beam to impinge on the at least one elevated light marker at at least one light beam impingement, respectively, when the at least one surgical equipment unit is at the at least one horizontal equipment operating position, respectively.

Illustrative embodiments of the disclosure are further generally directed to surgical patient and/or equipment alignment methods for optimal positioning of a patient and/or at least one surgical equipment unit for a surgical procedure in an operating room. An illustrative embodiment of the method may include at least one of the following: determining a horizontal patient operating position corresponding to an optimum position for a surgical patient table within a horizontal plane, emitting a downward light beam from a horizontal patient positioning light emitter directly above the horizontal patient operating position, placing the patent on a surgical patient table, moving the surgical patient table to the horizontal patient operating position by moving the surgical patient table within the horizontal plane until the downward light beam impinges at a selected point on the patient, and securing the surgical patient table at the horizontal patient operating position; and determining a horizontal equipment operating position corresponding to an optimum position for the at least one surgical equipment unit within the horizontal plane, emitting an upward light beam from a horizontal equipment positioning light emitter on the at least one surgical equipment unit, moving the at least one surgical equipment unit within the horizontal plane until the upward light beam impinges on an elevated light marker directly above the horizontal equipment operating position of the at least one surgical equipment unit, and securing the at least one surgical equipment unit at the horizontal equipment operating position.

Illustrative embodiments of the disclosure are further generally directed to surgical patient and/or equipment alignment methods for optimal positioning of any combination of at least two of a surgical patient table and at least one surgical equipment unit for a surgical procedure in an operating room. An illustrative embodiment of the method may include establishing a selected one of the at least two of the surgical patient table and the at least one surgical equipment unit as an anchor point for positioning of at least one remaining one of the at least two of the surgical patient table and the at least one surgical equipment unit by deploying the selected one of the at least two of the surgical patient table and the at least one surgical equipment unit at a corresponding selected one of a patient operating position and an equipment operating position, respectively, in the operating room; and deploying the at least one remaining one of the at least two of the surgical patient table and the at least one surgical equipment unit relative to the selected one of the at least two of the surgical patient table and the at least one surgical equipment unit at the corresponding selected one of the patient operating position and the equipment operating position, respectively, in the operating room.

Illustrative embodiments of the disclosure are further generally directed to emitter positioning assemblies. An illustrative embodiment of the emitter positioning assemblies may include vertical assembly arm. A horizontal assembly arm may be supported by the vertical assembly arm. A horizontal patient positioning light emitter may be supported by the horizontal assembly arm. The horizontal patient positioning light emitter may be configured to emit a downward light beam along a vertical Z axis.

### BRIEF DESCRIPTION OF THE DRAWINGS

Illustrative embodiments of the disclosure will now be described, by way of example, with reference to the accompanying drawings, in which:

FIG. 1 is a view of an operating room floor in an operating room in typical implementation of an illustrative embodiment of the surgical patient and/or equipment alignment systems of the disclosure, with multiple units of surgical equipment and a surgical patient table deployed in their respective horizontal equipment and horizontal patient operating positions after horizontal X and Y axis adjustment of the equipment and the surgical patient table and vertical Z axis adjustment of the surgical patient table;

FIG. 2 is a view of the operating room ceiling in the operating room illustrated in FIG. 1, with multiple elevated light markers corresponding to respective horizontal equipment operating positions of the surgical equipment and a horizontal patient positioning light emitter corresponding to the horizontal patient operating position of the surgical patient table in typical implementation of the illustrative surgical patient and/or equipment alignment system;

FIG. 3 is a front view of the operating room illustrated in FIG. 1 in typical implementation of the illustrative surgical patient and/or equipment alignment system;

FIG. 4 is an enlarged top sectional view of a portion of the operating room floor of the operating room, more particularly illustrating typical rotational X and Y axis positional adjustment of a unit of the surgical equipment in implementation of some embodiments of the surgical patient and/or equipment alignment systems;

FIG. 5 is an enlarged top sectional view of a portion of the operating room floor of the operating room, prior to typical horizontal X and Y axis positional adjustment of the surgical patient table to the horizontal patient operating position (illustrated in phantom);

FIG. 6 is an enlarged top sectional view of the operating room floor and the surgical patient table illustrated in FIG. 5, with the surgical patient table in the horizontal patient operating position after positional adjustment is completed;

FIG. 7 is a front sectional view of a portion of the operating room prior to vertical Z axis positional adjustment of the surgical patient table to the desired height, with a downward light beam with crosshairs emitted from the horizontal patient positioning light emitter (not illustrated) and impinging on the patient and an angular light beam with crosshairs emitted from the vertical patient positioning light emitter initially above and across the patient;

FIG. 8 is a front sectional view of the portion of the operating room illustrated in FIG. 7, with the surgical patient table adjusted to the desired height as the downward light beam with crosshairs emitted from the horizontal patient positioning light emitter impinges on the patient and the angular light beam with crosshairs emitted from the vertical patient positioning light emitter impinges on the patient in coinciding relationship to the crosshairs of the downward light beam;

FIG. 9 is a top sectional view of the surgical patient table with the crosshairs of the angular light beam emitted by the vertical patient positioning light emitter coinciding on the patient with the crosshairs of the downward light beam emitted by the horizontal patient positioning light emitter;

FIG. 10 is an enlarged view of the angular light beam crosshairs of the angular light beam coinciding on the patient with the downward light beam crosshairs of the downward light beam;

FIG. 11 is an enlarged top sectional view of a portion of the operating room floor of the operating room, prior to typical horizontal X and Y axis positional adjustment of a surgical equipment unit to the horizontal equipment operating position (illustrated in phantom);

FIG. 12 is an enlarged top sectional view of the operating room floor and the surgical equipment illustrated in FIG. 11, with the surgical equipment unit in the horizontal equipment operating position after positional adjustment is completed;

FIG. 13 is an enlarged top sectional view of a portion of the operating room floor and an operating room wall of the operating room, prior to typical horizontal rotational X and Y axis positional adjustment of a rotatable surgical equipment unit to a horizontal rotational position (illustrated in phantom);

FIG. 14 is an enlarged top sectional view of the operating room floor and operating room wall and the rotatable surgical equipment unit illustrated in FIG. 13, with the rotatable surgical equipment in the horizontal rotational position after positional adjustment is completed;

FIG. 15 is a top view of an operating room floor in an operating room in typical implementation of an illustrative ophthalmic surgical embodiment of the surgical patient and/or equipment alignment systems of the disclosure;

FIG. 16 is a bottom view of the operating room ceiling in the operating room illustrated in FIG. 15;

FIG. 17 is a perspective view of a typical ophthalmic operating microscope with a light emitter mount assembly and a light emitter retrofitted to the microscope in typical implementation of the system illustrated in FIG. 15;

FIG. 18 is an enlarged side view of the light emitter mount assembly illustrated in FIG. 17, mounted on the ophthalmic operating microscope (illustrated in section);

FIG. 19 is an enlarged sectional view of a portion of the ophthalmic operating microscope with the light emitter mount assembly built into the microscope according to some embodiments of the surgical patient and/or equipment alignment systems;

FIG. 20 is an enlarged top sectional view of the operating room floor and a side operating room wall of the operating room illustrated in FIG. 15, with a rotationally adjustable visualization system on the operating room floor, a rotational equipment position light emitter on the visualization system and a side light marker on the side operating room wall of the operating room;

FIG. 21 is an enlarged top sectional view of the operating room floor and side wall of the operating room illustrated in FIG. 15, with the display screen of the visualization system rotatably adjusted to the equipment operating position as a horizontal side light beam emitted by the rotational equipment position light emitter on the display screen is aligned with the side light marker on the OR wall;

FIG. 22 is a block diagram of an illustrative embodiment of the surgical patient and/or equipment alignment systems of the disclosure, more particularly illustrating a typical control system in which a centralized user interface is configured to operate the various components of the system;

FIG. 23 is a block diagram of an alternative illustrative embodiment of the surgical patient and/or equipment alignment systems of the disclosure, more particularly illustrating a typical control system in which an equipment user interface configured to control each surgical equipment unit may also be configured to operate the corresponding equipment positioning light emitter on the surgical equipment unit;

FIG. 24 is a block diagram of another alternative illustrative embodiment of the surgical patient and/or equipment alignment systems of the disclosure, more particularly illustrating a typical control system in which the equipment positioning light emitter on each unit of surgical equipment has its own dedicated light emitter user interface;

FIG. 25 is a flow diagram illustrating a typical method of assembling or setting up an illustrative embodiment of the surgical patient and/or equipment alignment systems;

FIG. 26 is a flow diagram of an illustrative embodiment of the surgical patient and/or equipment alignment methods of the disclosure;

FIG. 27 is a top view of an operating room floor in an operating room in typical implementation of an alternative illustrative embodiment of the surgical patient and/or equipment alignment systems of the disclosure, more particularly illustrating an emitter positioning assembly (illustrated in phantom) mounted on a surgical equipment unit and supporting and positioning the horizontal patient positioning light emitter (not illustrated) over the patient as the patient reclines on the surgical patient table to facilitate horizontal positioning of the patient in the operating room, with the emitter positioning assembly also supporting the vertical patient positioning light emitter as an angular light beam is emitted from the vertical patient positioning light emitter onto the patient to facilitate vertical positioning of the patient in the operating room;

FIG. 28 is a bottom view of the operating room ceiling in the operating room illustrated in FIG. 27, with the emitter positioning assembly supporting the horizontal patient positioning light emitter over the horizontal patient operating position in the operating room;

FIG. 29 is a rearward view of the operating room, taken along viewing lines 29-29 in FIG. 27, in typical implementation of the illustrative surgical patient and/or equipment alignment system, with the emitter positioning assembly supporting the horizontal patient positioning light emitter over the patient and a downward light beam emitted from the horizontal patient positioning light emitter onto the patient and supporting the vertical patient positioning light emitter with an angular light beam emitted from the vertical patient positioning light emitter onto the patient;

FIG. 30 is an enlarged sectional top view of a portion of the operating room floor of the operating room illustrated in FIG. 27, with the vertical patient positioning light emitter supported by the emitter positioning assembly, more particularly illustrating typical positioning of the horizontal patient positioning light emitter over the patient via operation of the emitter positioning assembly in implementation of some embodiments of the surgical patient and/or equipment alignment systems;

FIG. 31 is a front view of an operating room in which the surgical patient and/or equipment alignment systems includes a visualization system with the emitter positioning assembly mounted on the visualization system and the horizontal patient positioning light emitter supported by the emitter positioning assembly over the patient as the downward light beam is emitted from the horizontal patient positioning light emitter onto the patient, and the vertical patient positioning light emitter mounted on the emitter positioning assembly as the angular light beam is emitted from the vertical patient positioning light emitter onto the patient;

FIG. 32 is a top view of a portion of the operating room illustrated in FIG. 31 with the visualization system adjacent to the surgical patient table supporting the patient, a horizontal light beam emitted from the rotational equipment positioning light emitter and impinging on the side light marker and the emitter positioning assembly extending from the visualization system and supporting the horizontal patient positioning light emitter over the patient for horizontal positioning of the surgical patient table and the patient, and the vertical patient positioning light emitter mounted on the emitter positioning assembly;

FIG. 33 is a front view of an operating room in which the surgical patient and/or equipment alignment systems includes an emitter assembly stand with the emitter positioning assembly mounted on the emitter assembly stand, the horizontal patient positioning light emitter supported by the emitter positioning assembly over the patient as the downward light beam is emitted from the horizontal patient positioning light emitter onto the patient, and the vertical patient positioning light emitter supported by the emitter positioning assembly as the angular light beam is emitted from the vertical patient positioning light emitter onto the patient;

FIG. 34 is a flow diagram of another illustrative embodiment of the surgical patient and/or equipment alignment methods of the disclosure in which a surgical equipment unit serves as an anchor point for positioning of the surgical patient table and/or the remaining surgical equipment unit(s) in the operating room; and

FIG. 35 is a flow diagram of another illustrative embodiment of the surgical patient and/or equipment alignment methods of the disclosure in which the surgical patient table serves as an anchor point for positioning of the surgical equipment unit(s) in the operating room.

### DETAILED DESCRIPTION

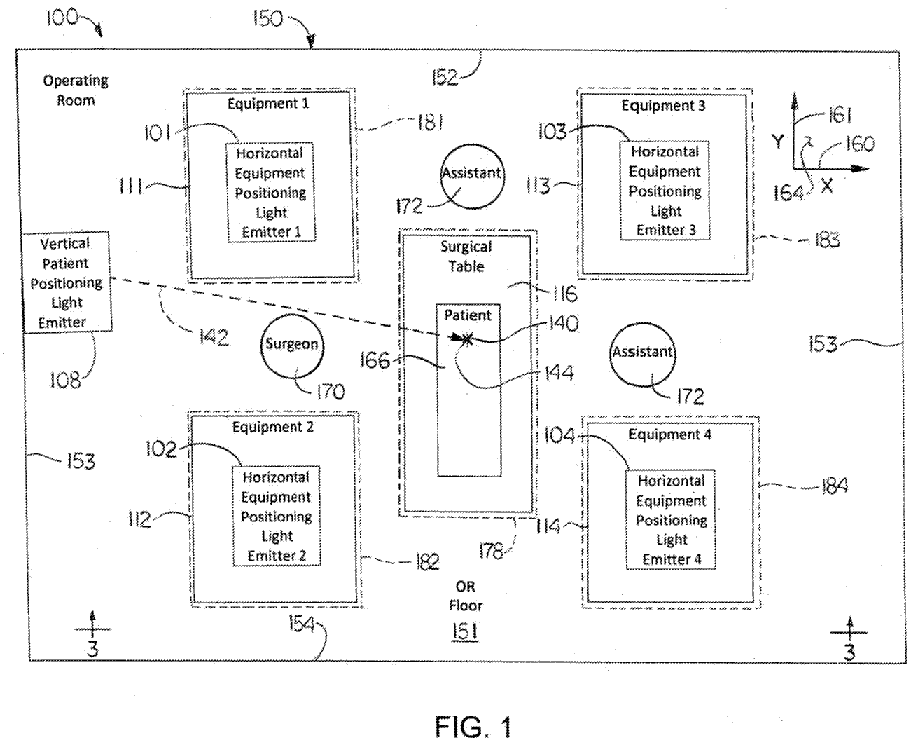

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

All methods set forth in the present disclosure may be performed in any suitable order of steps unless otherwise indicated herein or contradicted by the rules of logic. The use of any and all examples or exemplary language provided herein is intended to clearly describe the subject matter of the disclosure and is not intended to be limiting on the scope of the subject matter set forth in the claims. No element, step, ingredient, or limitation mentioned or described in the specification shall not be construed as regarding any unclaimed component, step, or limitation to be essential in practicing the claimed subject matter.

Unless expressly or implicitly indicated otherwise, throughout the description and the appended claims, the terms “comprise”, “comprising”, “comprised of” and the like are to be construed in an inclusive sense as opposed to an exclusive or exhaustive sense, and are equivalent to the phrase, “including but not limited to”. Each embodiment disclosed herein can comprise, consist essentially of, or consist of its particular stated element, step, ingredient, or limitation. As used herein, the transition term “comprise” or “comprises” means “includes, but is not limited to, and allows for the inclusion of unspecified elements, steps, ingredients, or limitations, even in major amounts”. The transitional phrase “consisting of” excludes any element, step, ingredient, or limitation not specified. The transition phrase “consisting essentially of” shall limit the scope of the embodiment to the specified elements, steps, ingredients, or limitations and to those that do not materially affect the embodiment. Throughout the written description, drawings and claims appended hereto, unless otherwise noted, it shall be recognized and understood that each embodiment of the described, illustrated and claimed subject matter may comprise, consist essentially of, or consist of any component, element or combination of components or elements set forth herein.

Unless otherwise noted using precise or limiting terminology, all numbers which express quantities of ingredients throughout the specification and claims are to be understood as being approximations of the numerical value cited to express the quantities of those ingredients. As used throughout the specification and claims, the term “about” has the meaning reasonably ascribed to it by a person skilled in the art when used in conjunction with a stated numerical value or range, i.e., denoting from the exact stated value or range to somewhat more or somewhat less than the stated value or range, from a deviation of from 0% with respect to the stated value or range to up to and including 20% of the stated value or range in either direction.

Various illustrative embodiments of the disclosure are described herein. Variations on the described illustrative embodiments may become apparent to those of ordinary skill in the art in reading the specification, drawings and claims of the disclosure. Accordingly, the disclosure encompassed by the specification, claims and drawings includes all modifications and equivalents of the subject matter recited in the claims as permitted by applicable law. Additionally, any combination of the elements in all possible variations thereof is encompassed by the subject matter of the disclosure unless otherwise indicated herein.

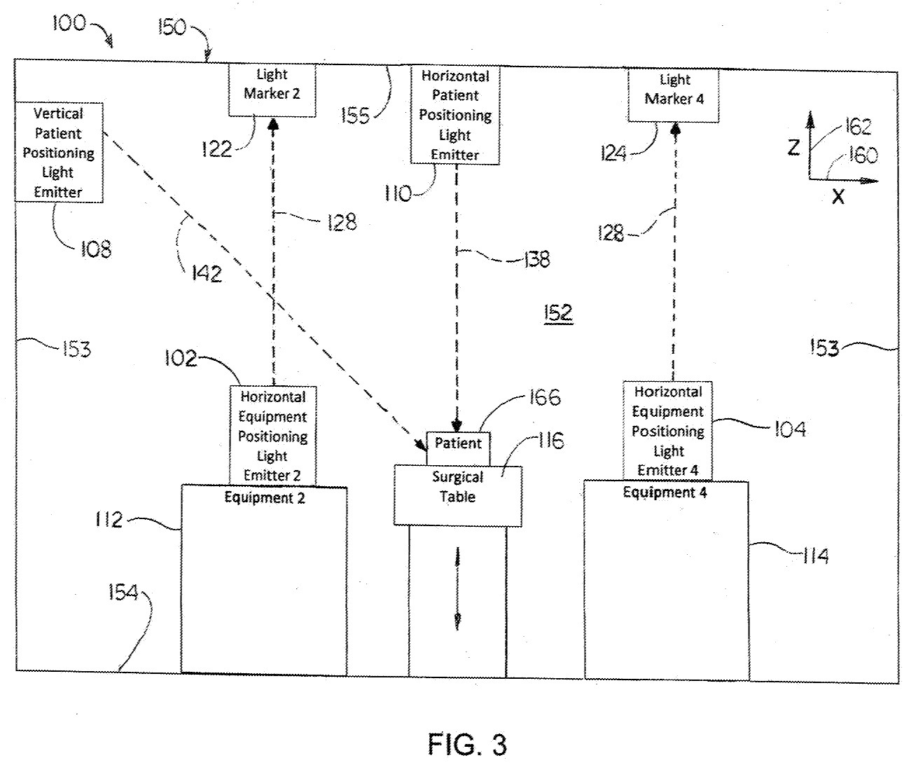

Referring initially to FIGS. 1-14 of the drawings, an illustrative embodiment of the surgical patient and/or equipment alignment systems, hereinafter system, of the disclosure is generally indicated by reference numeral 100. As illustrated in FIG. 1 and will be hereinafter described, in typical application, the system 100 may facilitate quick and precise positioning of a surgical table or surgical patient table 116 on which a patient 166 reclines within a horizontal plane 164 formed by a horizontal X axis 160 and a horizontal Y axis 161 for a surgical procedure in an operating room (OR) 150. As illustrated in FIG. 3, in some embodiments, the system 100 may facilitate quick and precise vertical positioning of the surgical patient table 116 along a Z axis 162 for positioning of the patient 166 at a selected height for the surgical procedure. As used herein, “operating room” includes but is not limited to any room, enclosure, area or space in a clinic, hospital, medical center or other medical facility which is dedicated to or suitable for implementation of medical procedures.

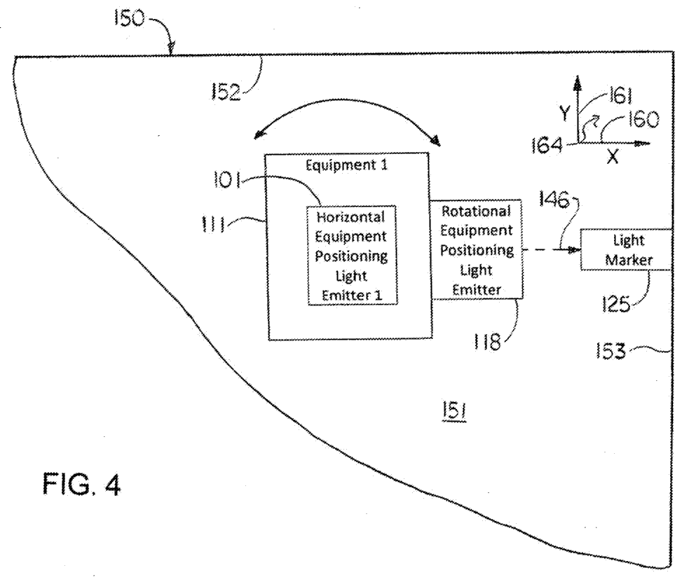

As further illustrated in FIG. 1, in some embodiments, the system 100 may facilitate quick and precise horizontal positioning of at least one surgical equipment unit 111, 112, 113, 114 within the horizontal plane 164 formed by the X axis 160 and the Y axis 161 for the surgical procedure. As illustrated in FIG. 4, in some embodiments, the system 100 may facilitate horizontal rotational positioning of at least one of the surgical equipment units 111, 112, 113, 114 within the horizontal plane 164. Precise horizontal positioning of the patient 166 and/or the surgical equipment unit or units 111, 112, 113, 114 may be necessary or desirable for a surgeon 170 and/or one or more surgical assistants 172 (FIG. 1) to implement the surgical procedure in a precise, efficient, and time-effective manner. While the non-limiting example of the system 100 is shown and described herein as having four surgical equipment units 111, 112, 113 and 114, respectively, it will be recognized and understood that the system 100 may be implemented using any number of the surgical equipment units.

As illustrated in FIG. 3 and will be hereinafter described, the system 100 may utilize at least one downward light beam 138 to facilitate positioning of the surgical patient table 116 within the horizontal plane 164 (FIG. 1). The system 100 may utilize at least one angular light beam 142 to facilitate the vertical positioning of the surgical patient table 116 along the Z axis 162. The angular light beam 142 may be oriented downwardly at an angle with respect to the horizontal plane 164 formed by the X axis 160 and the Y axis 161. The system 100 may utilize at least one upward light beam 128 to facilitate the horizontal positioning of the surgical equipment 111, 112, 113, 114 within the horizontal plane 164. As illustrated in FIG. 4, the system 100 may utilize at least one typically horizontal side light beam 146 to facilitate the horizontal rotational positioning of one or more units of the surgical equipment 111, 112, 113, 114 within the horizontal plane 164.

As illustrated in FIGS. 1-3, the OR 150 in which the system 100 is implemented may have a standard or conventional operating room design or layout, typically with an OR floor 151; a rear OR wall 152; side OR walls 153; a front OR wall 154; and an OR ceiling 155. As illustrated in FIG. 1, the surgical patient table 116 which supports the patient 166 may have an optimum horizontal patient operating position 178 on the OR floor 151. Additionally or alternatively, each unit of the surgical equipment 111, 112, 113, 114 may have a corresponding optimal horizontal equipment operating position 181, 182, 183, 184 on the OR floor 151. The optimum horizontal patient operating position 178 for the surgical patient table 116 and/or the optimum horizontal equipment operating position or positions 181, 182, 183, 184 for the respective unit or units of the surgical equipment 111, 112, 113, 114 may be necessary or desirable for the surgeon 170 and/or the surgical assistant or assistants 172 to easily access, manipulate and operate the surgical equipment 111, 112, 113, 114 for precise, efficient, and time-effective management and implementation of the procedure. Accordingly, the system 100 may facilitate quick and precise return or repositioning and orientation of the surgical patient table 116 and/or the surgical equipment 111, 112, 113, 114 to the respective optimum horizontal patient operating position 178 and horizontal equipment operating position or positions 181, 182, 183, 184 after removal of the surgical patient table 116 and/or the surgical equipment 111, 112, 113, 114 from the respective optimum horizontal patient and equipment operating positions, particularly in the event that the surgical patient table 116 and/or the surgical equipment 111, 112, 113, 114 is moved in or removed from the operating room 150 for cleaning of the operating room 150 between procedures, or for equipment repair, maintenance, replacement and/or other purposes.

The system 100 may be adaptable to any type of surgical or medical procedure in which precise positioning of the patient 166 via the surgical patient table 116 and/or the surgical equipment 111, 112, 113, 114 in the OR 150 along the X axis 160 and the Y axis 161 within the horizontal plane 164 and/or along the vertical Z axis 162 is desired. Surgical or medical procedures which may be suitable for implementation of the system 100 may include but are not limited to general, cardiac, thoracic, urological, otorhinolaryngological, orthopedic, podiatric, plastic or cosmetic, ophthalmic, and neurosurgical surgical and medical procedures, as well as invasive and non-invasive cardiology and other medical procedures. The surgical equipment 111, 112, 113, 114 which is used to carry out the surgical procedure may depend on the procedure involved. Non-limiting examples of the surgical equipment may include surgical robotic equipment, instrument holding equipment, anesthesia equipment, surgical microscopes, vitrectomy equipment, visualization equipment, and the like.

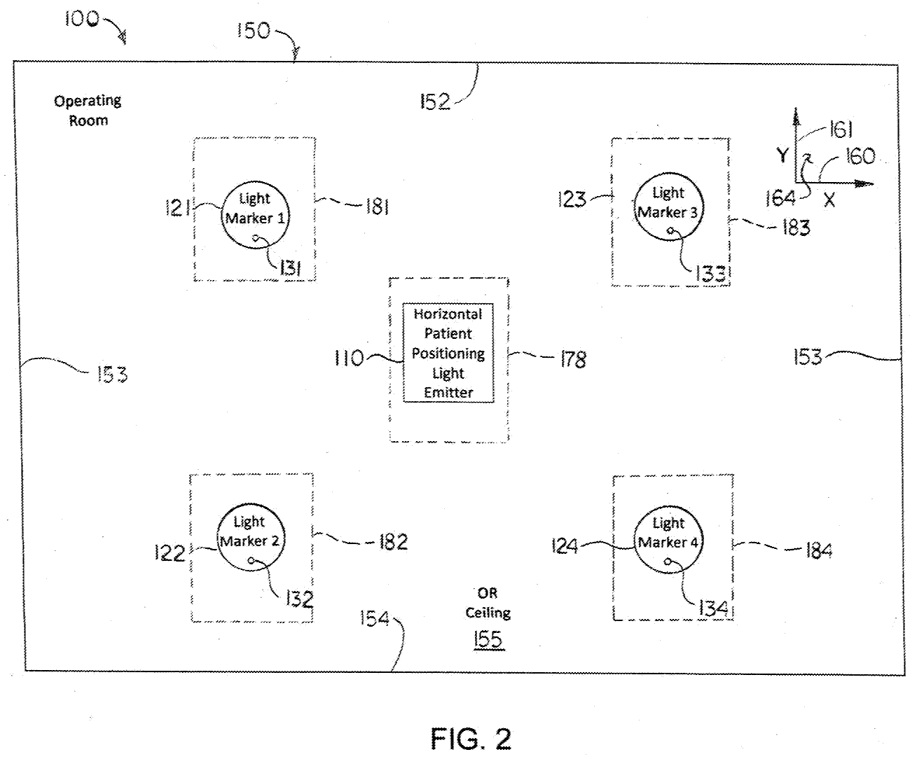

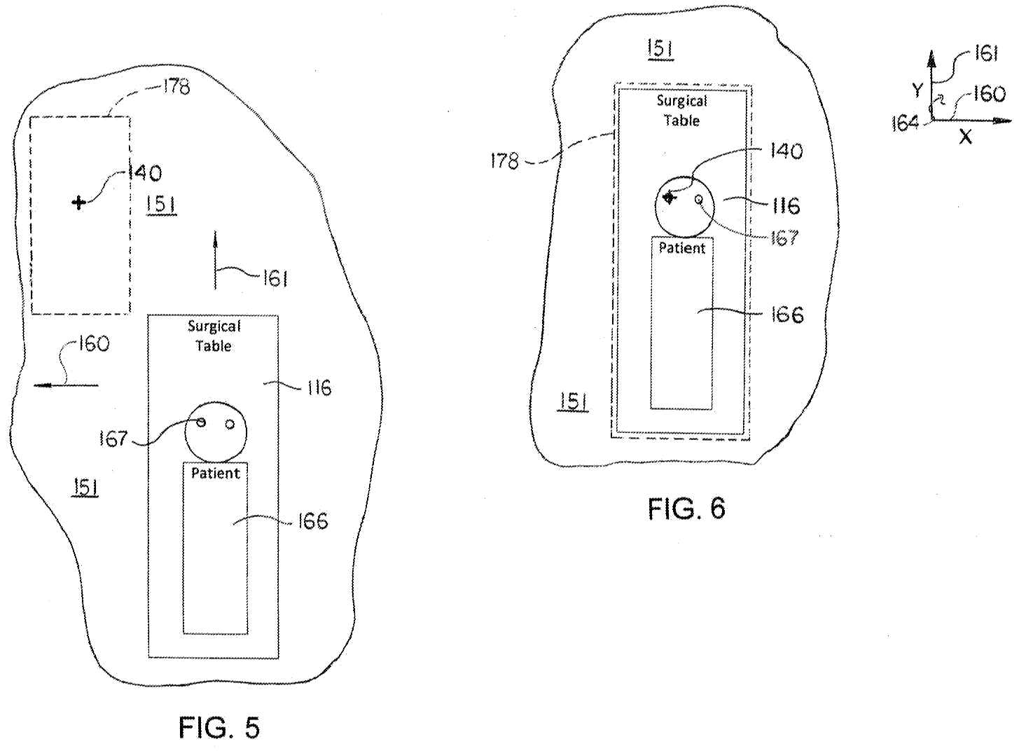

As illustrated in FIG. 2, at least one horizontal patient positioning light emitter 110 may be provided on the OR ceiling 155. The horizontal patient positioning light emitter 110 may be positioned directly above the horizontal patient operating position 178 which defines the optimum operating position of the surgical patient table 116 on the OR floor 151. The horizontal patient positioning light emitter 110 may include any type of device or combination of devices configured to emit the downward light beam 138 (FIG. 3) toward and against the patient 166 as the patient 166 reclines on the surgical patient table 116. In some embodiments, the horizontal patient positioning light emitter 110 may include at least one optical projector, for example and without limitation. The optical projector or projectors of the horizontal patient positioning light emitter 110 may be configured to generate and emit the downward light beam 138 (FIG. 3) such that the downward light beam 138 impinges on the patient 166 as the patient 166 reclines on the surgical patient table 116. The downward light beam 138 may have an intensity which is safe to the eyes 167 (FIG. 5) of the patient 166. As illustrated in FIGS. 1 and 6, the downward light beam 138 may be configured to form downward light beam crosshairs 140 at its point of impingement on the patient 166. The downward light beam crosshairs 140 may mark a surgical site on the patient 166.

As illustrated in FIG. 1, in some embodiments, at least one vertical patient positioning light emitter 108 may be provided on the rear OR wall 152, one of the side OR walls 153 (as illustrated), the OR ceiling 155 and/or on the front OR wall 154. The vertical patient positioning light emitter 108 may be configured to emit the angular light beam 142 at a downward angle. The vertical patient positioning light emitter 108 may include any type of device or combination of devices configured to emit the angular light beam 142 toward and against the patient 166 when the surgical patient table 116 is deployed at the optimal height for the surgical procedure as the patient 166 reclines on the surgical patient table 116. In some embodiments, the vertical patient positioning light emitter 108 may include at least one optical projector, for example and without limitation. The optical projector or projectors of the vertical patient positioning light emitter 108 may be configured to generate and emit the angular light beam 142 such that the angular light beam 142 impinges on the patient 166 as the patient 166 reclines on the surgical patient table 116. The angular light beam 142 may have an intensity which is safe to the eyes 167 of the patient 166.

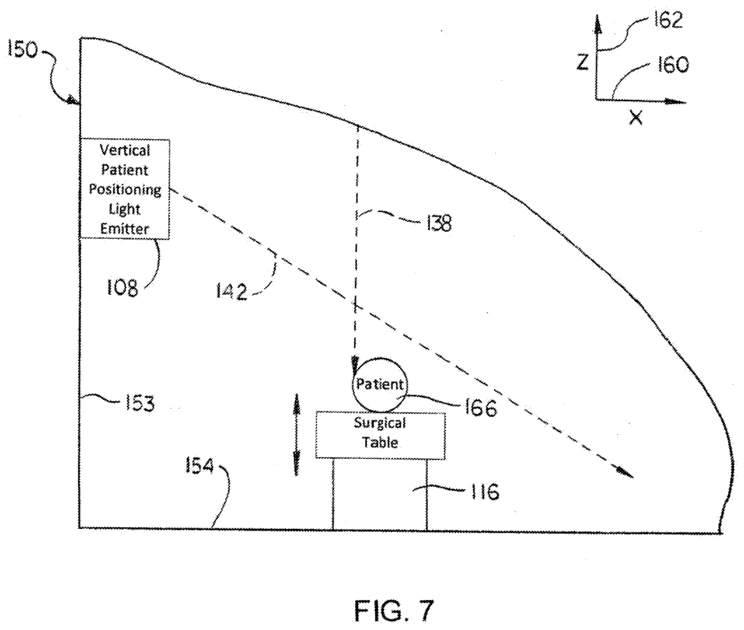

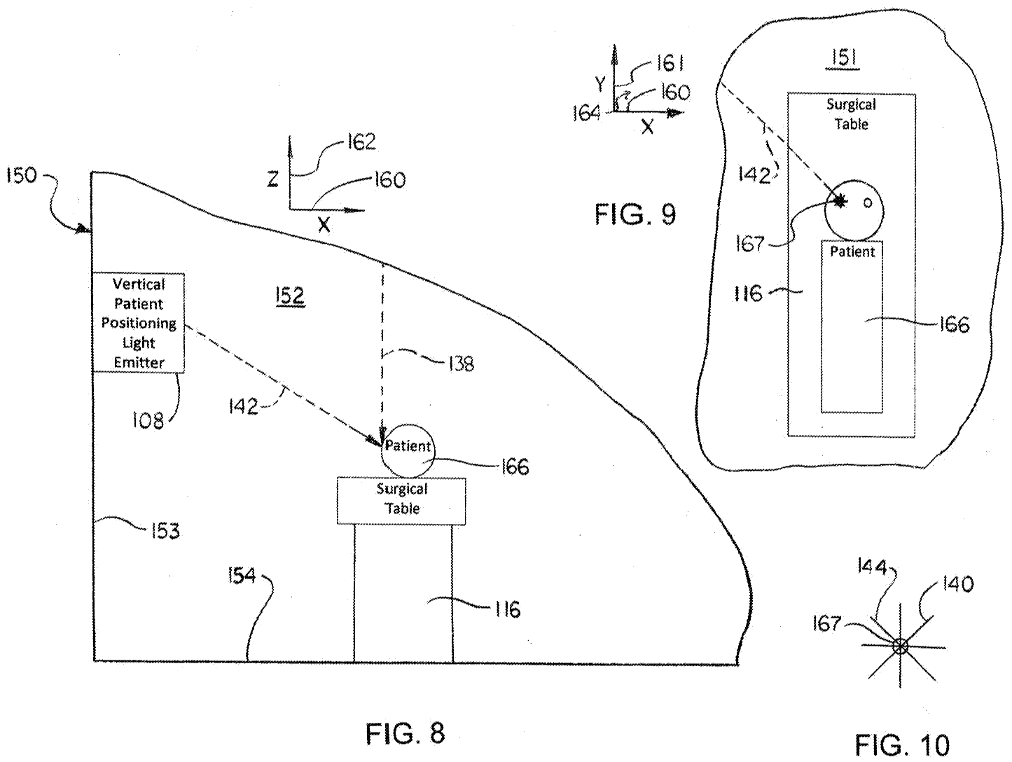

As illustrated in FIG. 7, the vertical patient positioning light emitter 108 may be oriented such that the angular light beam 142 traverses over and across the patient 166 when the surgical patient table 116 supports the patient 116 beneath the desired operating height or vertical operating position above the OR floor 154 and impinges on the patient 166 when the surgical patient table 116 supports the patient 166 at the selected vertical operating position (FIG. 8). As illustrated in FIGS. 9 and 10, the angular light beam 142 may be configured to form angular light beam crosshairs 144 at its point of impingement on the patient 166. As illustrated in FIG. 10, the angular light beam crosshairs 144 of the angular light beam 142 may coincide or intersect with the downward light beam crosshairs 140 of the downward light beam 138 at the points of impingement of the respective downward light beam 138 and angular light beam 142 on the patient 166.

As further illustrated in FIG. 1, in some embodiments, at least one horizontal equipment positioning light emitter 101, 102, 103, 104 may be provided on each corresponding surgical equipment unit 111, 112, 113, 114. As illustrated in FIG. 3, each horizontal equipment positioning light emitter 101, 102, 103, 104 may be configured to emit a corresponding upward light beam 128. In some embodiments, each horizontal equipment positioning light emitter 101, 102, 103, 104 may include a laser which is configured to emit a collimated laser beam as the upward light beam 128.

As illustrated in FIG. 2, elevated light markers 121, 122, 123, 124 may be provided in the OR 150 directly above the horizontal equipment operating positions 181, 182, 183, 184 of the respective surgical equipment units 111, 112, 113, 114. For example and without limitation, in some embodiments, the elevated light markers 121, 122, 123, 124 may be provided on the OR ceiling 155. In other embodiments, the elevated light markers 121, 122, 123, 124 may additionally or alternatively be provided on one or more elevated structures beneath the OR ceiling 155 in the OR 150. When the surgical equipment units 111, 112, 113, 114 are in their respective horizontal equipment operating positions 181, 182, 183, 184, the upward light beams 128 emitted by the respective horizontal equipment positioning light emitters 101, 102, 103, 104 may positionally coincide with and form light beam impingements 131, 132, 133, 134 on the respective elevated light markers 121, 122, 123, 124.

Each elevated light marker 121, 122, 123, 124 may include any type of marker having a color, texture, and/or other appearance which visually contrasts with the OR ceiling 155 and/or other elevated structure on which the elevated light markers are placed. For example and without limitation, each elevated light marker 121, 122, 123, 124 may include an ink, paint or chalk spot or mark, protrusion, or indentation, or a button, tab, disk, strip, or tape made of plastic, metal, fabric, and/or other materials. In some embodiments, the upward light beams 128 emitted by the horizontal equipment positioning light emitters 101, 102, 103, 104 may be color-coded to match colors assigned to the respective surgical equipment units 111, 112, 113, 114. For example and without limitation, the upward light beams 128 may be blue, green, yellow, and red to identify which surgical equipment unit 111, 112, 113, 114 corresponds to which elevated light marker 121, 122, 123, 124. Accordingly, each elevated light marker 121, 122, 123, 124 may be color-coded to match the color of the upward light beam 128 emitted by its corresponding horizontal equipment positioning light emitter 101, 102, 103, 104. In some embodiments, the surgical equipment units 111, 112, 113, 114 may be assigned with numbers, letters, and/or other identifying indicia, in which case the elevated light markers 121, 122, 123, 124 which correspond to the horizontal equipment operating positions 181, 182, 183, 184 of the respective surgical equipment units 111, 112, 113, 114 may have the same matching number, letter, and/or other identifying indicia.

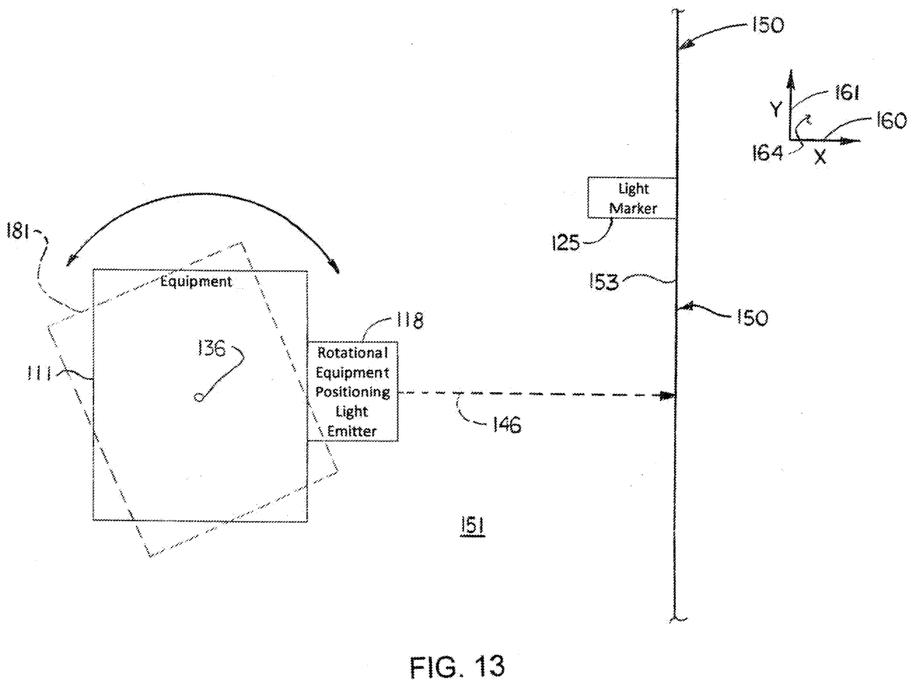

As illustrated in FIGS. 4, 13 and 14, in some embodiments, the optimal horizontal equipment operating position 181 or an optimum horizontal rotational position of a surgical equipment unit 111 or a component of the surgical equipment unit 111 may be achieved by rotating the surgical equipment unit 111 or the component of the surgical equipment unit 111 about a rotational axis 136 (FIG. 13) which may coincide with the vertical Z axis 162. For example and without limitation, the surgical equipment unit 111 may include a display screen of a visualization system (not illustrated), which will be hereinafter described. The display screen may require a particular rotational orientation with respect to the surgeon 170 (FIG. 1) for optimal viewing and visualization capabilities of the surgeon 170 during the surgical procedure. Accordingly, at least one rotational equipment positioning light emitter 118 may be provided on the surgical equipment unit 111. The rotational equipment positioning light emitter 118 may be configured to emit a typically horizontal side light beam 146. In some embodiments, the rotational equipment positioning light emitter 118 may include a laser which is configured to emit a collimated laser beam as the side light beam 146.

At least one side light marker 125 may be provided on the rear OR wall 152, a side OR wall 153 (as illustrated), and/or on the front OR wall 154. The location or position of the side light marker 125 may be selected such that the side light beam 146 emitted by the rotational equipment positioning light emitter 118 impinges on the side light marker 125 when the surgical equipment unit 111 or its rotatable component is in the selected rotated optimum horizontal rotational position within the horizontal plane 164 formed by the X axis 160 and the Y axis 161. The side light marker 125 may have the same design as that which was heretofore described for the elevated light markers 121, 122, 123, 124.

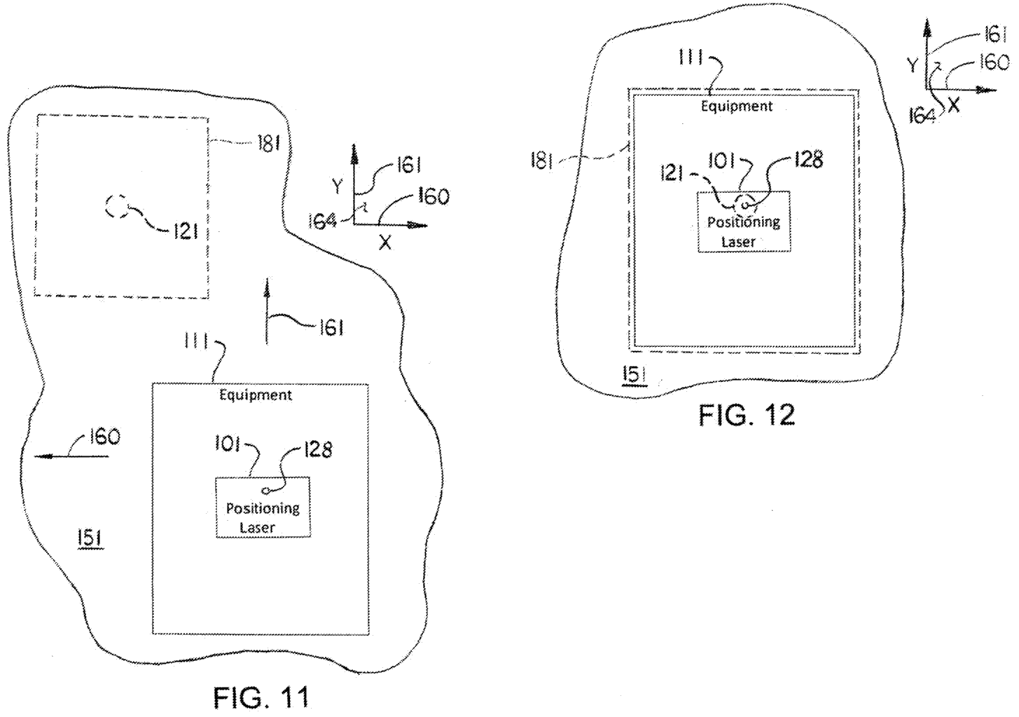

In typical application of the system 100, one or more of the surgical equipment units 111, 112, 113, 114 and the surgical patient table 116 may have been moved from its corresponding operating position 181, 182, 183, 184 for cleaning of the OR 150 and/or for equipment maintenance, repair, replacement and/or other purposes. Accordingly, the horizontal equipment light emitters 101, 102, 103, 104 may be operated to emit the respective upward light beams 128 initially typically against the OR ceiling 155. As illustrated in FIG. 11, each surgical equipment unit 111, 112, 113, 114 may be horizontally pulled, pushed and/or otherwise maneuvered on the OR floor 151 along the horizontal X-axis 160 and/or the horizontal Y-axis 161 within the horizontal plane 164 until the upward light beams 128 positionally coincide with and impinge on the respective elevated light markers 121, 122, 123, 124 at the light beam impingements 131, 132, 133, 134 (FIG. 2). As illustrated in FIG. 12, each surgical equipment unit 111, 112, 113, 114 is thus located at its corresponding optimum horizontal equipment operating position 181, 182, 183, 184 on the OR floor 151 in preparation for implementation of the surgical procedure.

The patient 166 may be rolled into the OR 150 on a patient transport gurney (not illustrated) and then transferred onto the surgical patient table 116. The horizontal patient positioning light emitter 110 (FIG. 2) may be operated to emit the downward light beam 138 (FIG. 3) typically with the downward light beam crosshairs 140 initially impinging on the OR floor 151 at the patient operating position 178, as illustrated in FIG. 5. The surgical patient table 116 may be horizontally pulled, pushed and/or otherwise maneuvered on the OR floor 151 along the horizontal X-axis 160 and/or the horizontal Y-axis 161 within the horizontal plane 164 until the downward light beam crosshairs 140 positionally coincide with and impinge on the surgical site on the patient 166. As illustrated in FIG. 6, the surgical patient table 116 is thus located at the optimum horizontal patient operating position 178 on the OR floor 151 in preparation for implementation of the surgical procedure. The horizontal equipment positioning light emitters 101, 102, 103, 104 and the horizontal patient positioning light emitter 110 may then be turned off and the surgical procedure commenced.

As illustrated in FIGS. 7-10, in some applications, the system 100 may facilitate vertical height adjustment of the surgical patient table 116 along the Z axis 162 for proper positioning of the patient 166 at the desired height for the surgical procedure. Accordingly, the vertical patient positioning light emitter 108 may be operated to emit the angular light beam 142. As illustrated in FIG. 7, the angular light beam 142 may initially traverse above the patient 166 to typically impinge on and form the angular light beam crosshairs 144 (FIG. 10) on the OR floor 154. The surgical patient table 116 may then be operated to raise the patient 166 until the angular light beam 142 impinges on the patient 166, as illustrated in FIG. 8, and the angular light beam crosshairs 144 typically coincide with or intersect the downward light beam crosshairs 140 of the downward light beam 138 emitted by the horizontal patient positioning light emitter 110. The surgical patient table 116 may then be locked in place or otherwise secured at the selected height, after which the vertical patient positioning light emitter 108 may be turned off for commencement of the surgical procedure.

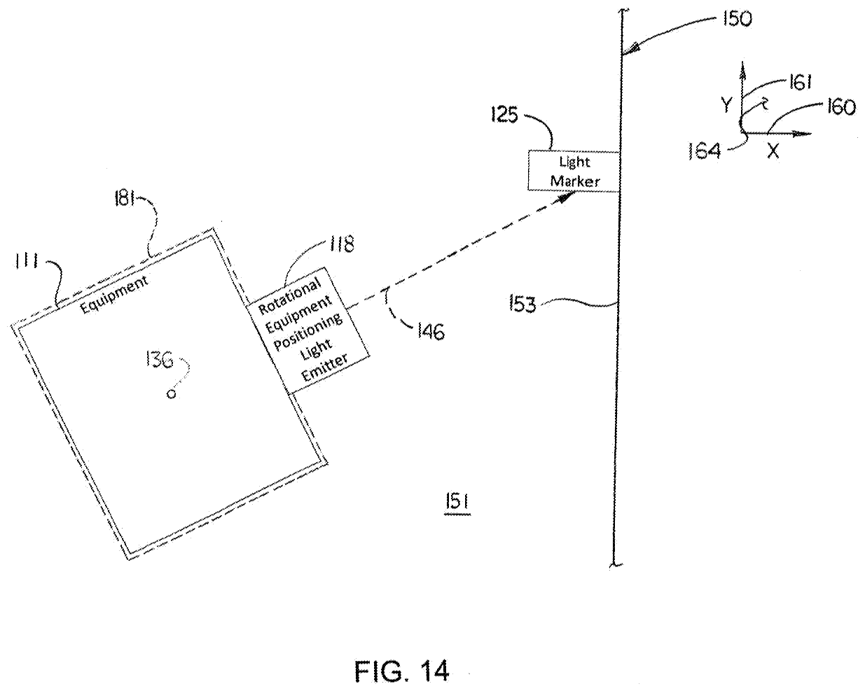

As illustrated in FIGS. 13 and 14, in some applications, the system 100 may facilitate rotational positional adjustment of one or more of the surgical equipment units 111, 112, 113, 114 within the horizontal plane 164. Accordingly, the rotational equipment positioning light emitter 118 may be operated to emit the side light beam 146. As illustrated in FIG. 13, the side light beam 146 may initially impinge on the side OR wall 153 adjacent to the side light marker 125. The surgical equipment unit 181 may then be rotated abouts its rotational axis 136 until the side light beam 146 impinges on the side light marker 125 and the surgical equipment unit 181 is thus oriented in the optimum horizontal equipment operating position 181, as illustrated in FIG. 14. The surgical equipment unit 181 may then be locked in place or otherwise secured and the rotational equipment positioning light emitter 118 turned off for commencement of the surgical procedure.

In some applications, the system 100 may be implemented in such a manner that a selected one of the surgical equipment units 111, 112, 113, 114 serves as an anchor point for subsequent positioning of the surgical patient table 116 and/or the remaining surgical equipment units 111, 112, 113, 114 in the operating room 150. For example and without limitation, the first surgical equipment unit 111 may initially be deployed in its first equipment operating position 181 typically via impingement of the upward light beam 128, emitted from the first horizontal equipment positioning light emitter 101, on the first light marker 121 at the first light beam impingement 131. Rotational adjustments of the first surgical equipment unit 111 may additionally be made such as by impingement of the side light beam 146 emitted by the rotational equipment positioning light emitter 118 against the side light marker 125, typically as was heretofore described. The first surgical equipment unit 111 would then be the anchor point for subsequent positioning of the surgical patient table 116, typically with the patient 166 resting thereon, at the patient operating position 178. Accordingly, the surgical patient table 116 may then be positioned relative to the first surgical equipment unit 111, typically by using the horizontal patient positioning light emitter 110, or by using any other suitable means, such as by estimating or otherwise determining the location of the patient operating position 178 relative to the first surgical equipment unit 111 and then rolling the surgical patient table 116 on the OR floor 151 to the patient operating position 178 and securing the surgical patient table 116 in place, for example and without limitation. The remaining surgical equipment units 112, 113, 114 may then be positioned relative to the first surgical equipment unit 111 or the surgical patient table 116, typically by using the respective light markers 122, 123, 124 or by using any other suitable means, such as by estimating or otherwise determining the locations of the equipment operating positions 182, 183, 184 relative to the first surgical equipment unit 111 or the surgical patient table 116, and then rolling the surgical equipment units 112, 113, 114 on the OR floor 151 to the respective equipment operating positions 182, 183, 184 and securing them in place, for example and without limitation.

In some applications, the system 100 may be implemented in such a manner that the surgical patient table 116 serves as an anchor point for subsequent positioning of one or more of the surgical equipment units 111, 112, 113, 114 at the respective equipment operating position(s) 181, 182, 183, 184. For example and without limitation, the surgical patient table 116, typically with the patient 166 resting thereon, may initially be deployed in the patient operating position 178 as it is guided into place typically via impingement of the downward light beam crosshairs 140 of the downward light beam 138 which is emitted by the horizontal patient positioning light emitter 110 on the patient 166, as heretofore described. The surgical patient table 116 would then be the anchor point for subsequent positioning of one or more of the surgical equipment units 111, 112, 113, 114. The surgical equipment unit(s) 111, 112, 113, 114 may then be positioned relative to the surgical patient table 116 in the respective equipment operating position(s) 181, 182, 183, 184 typically by rolling or guiding the unit(s) into place using the respective light markers 121, 122, 123, 124 as was heretofore described, or by using any other suitable means, such as by estimating or otherwise determining the locations of the equipment operating positions 182, 183, 184 151 relative to the surgical patient table 116 and then rolling or guiding the surgical equipment units 112, 113, 114 on the OR floor 151 to the respective equipment operating positions 181, 182, 183, 184, for example and without limitation.

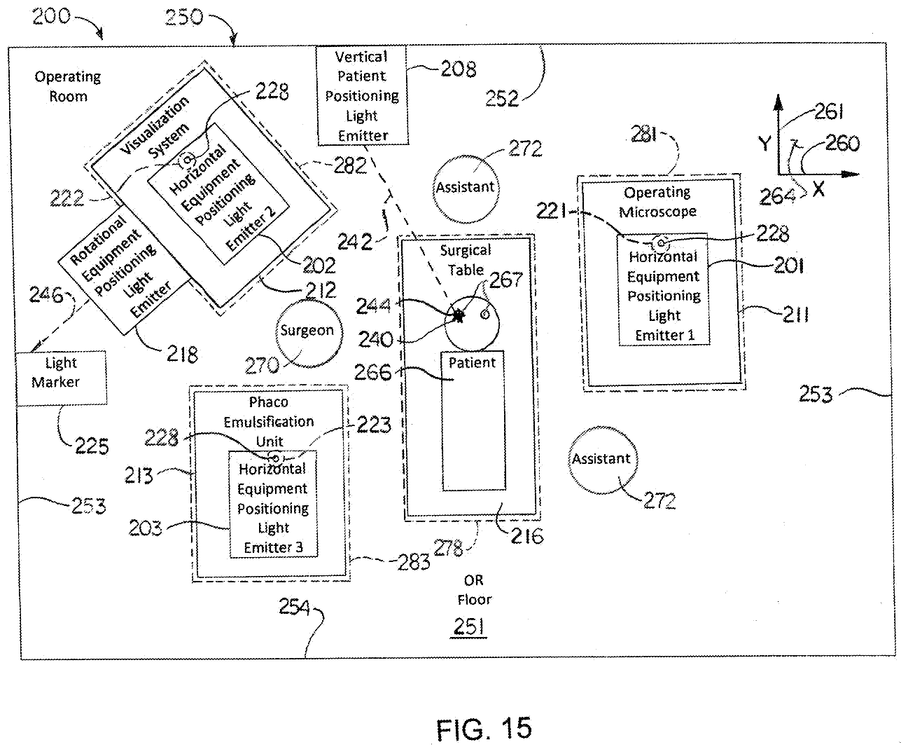

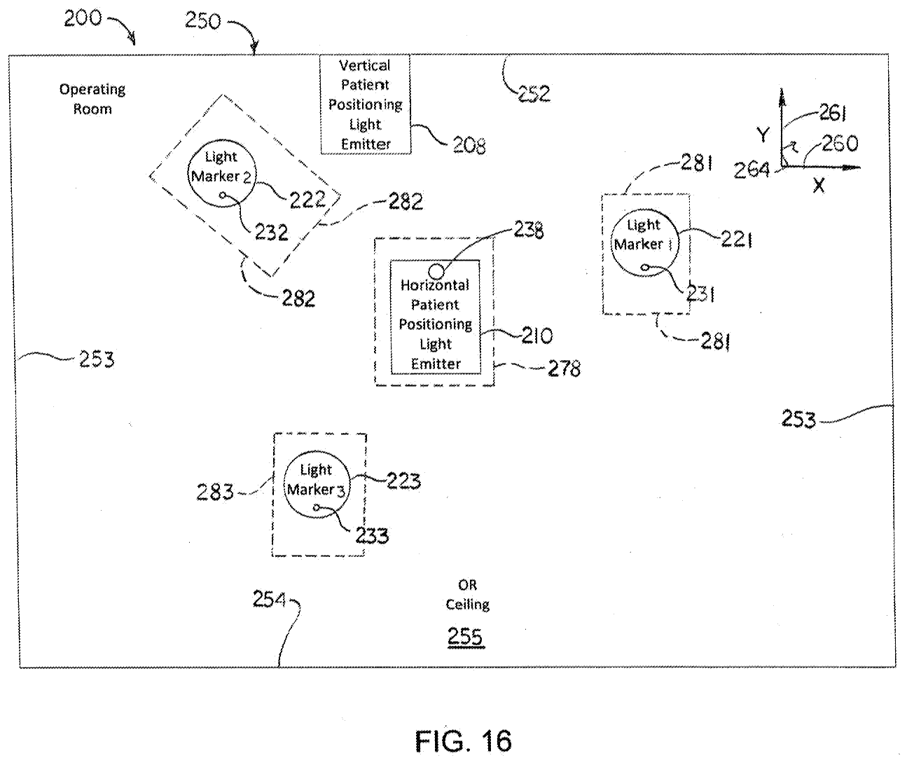

Referring next to FIGS. 15-21 of the drawings, an illustrative ophthalmic surgical embodiment of the surgical patient and/or equipment alignment systems of the disclosure is generally indicated by reference numeral 200. In the system 200, elements which are analogous to the respective elements of the system 100 that was heretofore described with respect to FIGS. 1-14 are designated by the same respective numerals in the 200-299 series in FIGS. 15-21. Thus, to the extent which is applicable, the same description which was heretofore described with respect to the system 100 is incorporated by reference herein in its entirety herein with respect to the system 200. Accordingly, in right-eye ophthalmic surgical procedures, the position of the horizontal patient positioning light emitter 210 (FIG. 16) on the OR ceiling 255 or other elevated structure in the operating room 150 may correspond to the position of the right eye 267 (FIG. 15) of the patient 266 as the patient 266 reclines on the surgical patient table 216 at the horizontal patient operating position 278. The downward light beam 238 (FIG. 16) emitted by the horizontal patient positioning light emitter 210 may thus impinge on the right eye 267 of the patient 266 with the downward light beam crosshairs 240 typically appearing over the patient's right eye 267. In left-eye procedures, the horizontal patient positioning light emitter 210 (FIG. 16) may correspond in position to the position of the left eye of the patient 266.

The vertical patient positioning light emitter 208 may be provided on the rear OR wall 252, as illustrated, or alternatively, on one of the side OR walls 253, the front OR wall 254 or the OR ceiling 255. The vertical patient positioning light emitter 208 may be oriented or aimed such that the angular light beam 242 emitted thereby impinges on the right eye 267 of the patient 266 when the surgical patient table 216 is positioned at the desired vertical operating position along the vertical Z axis 262. The angular light beam crosshairs 244 of the angular light beam 242 may thus coincide with or intersect the downward light beam crosshairs 240 of the downward light beam 238 (FIG. 16) emitted by the horizontal patient positioning light emitter 210.

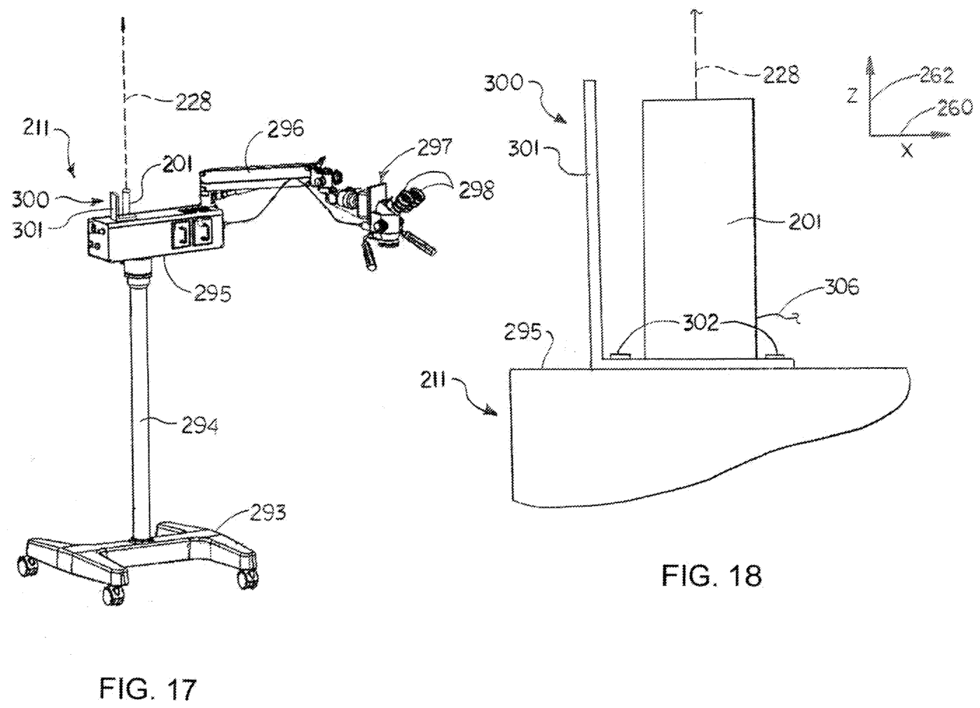

As illustrated in FIGS. 15 and 17-19, some ophthalmic surgical procedures may utilize an operating microscope 211. As illustrated in FIGS. 17-19, the operating microscope 211 may have a standard or conventional ophthalmic surgical microscope design with a typically wheeled base 293 which may portably support the operating microscope 211 on the OR floor 251. A post 294 may extend upwardly from the base 293. A main housing 295 may be supported by the post 294. The main housing 295 may house various operational and control features and components of the operating microscope 211. An elongated pivoting arm 296 may extend from the main housing 295. An ocular head 297 having eyepieces 298 may be supported by the arm 296.

As illustrated in FIG. 15, the operating microscope 211 may have a microscope operating position 281 which may be adjacent to the surgical patient table 216 on the OR floor 251. A horizontal equipment positioning light emitter 201 may be provided on the operating microscope 211, typically as will be hereinafter described. The horizontal equipment positioning light emitter 201 may be oriented upwardly to emit an upward light beam 228. As illustrated in FIG. 16, a first elevated light marker 221 may be provided on the OR ceiling 255 or other elevated structure in the operating room 250 directly above the microscope operating position 281. Accordingly, when the operating microscope 211 is in the microscope operating position 281, as illustrated in FIG. 15, the upward light beam 228 emitted by the horizontal equipment positioning light emitter 201 may positionally coincide with and impinge on the first elevated light marker 221 at a first light beam impingement 231, as illustrated in FIG. 16.

As illustrated in FIGS. 17 and 18, in some embodiments, the horizontal equipment positioning light emitter 201 may be retrofitted to the operating microscope 211. Accordingly, a light emitter mount assembly 300 which is suitable for the purpose may mount the horizontal equipment positioning light emitter 201 typically on the main housing 295 or an alternative component of the operating microscope 211. In some embodiments, the light emitter mount assembly 300 may include a mount bracket 301. Mount bracket fasteners 302 may attach the mount bracket 301 to the main housing 295. The horizontal equipment positioning light emitter 201 may be attached to the mount bracket 301 in an upwardly oriented position. In alternative embodiments, the light emitter mount assembly 300 may have other designs suitable for the purpose of mounting the horizontal equipment positioning light emitter 201 on the main housing 295 of the operating microscope 211. Wiring 306 may connect the horizontal equipment positioning light emitter 201 to a suitable power source and user interface (not illustrated), which will be hereinafter described.

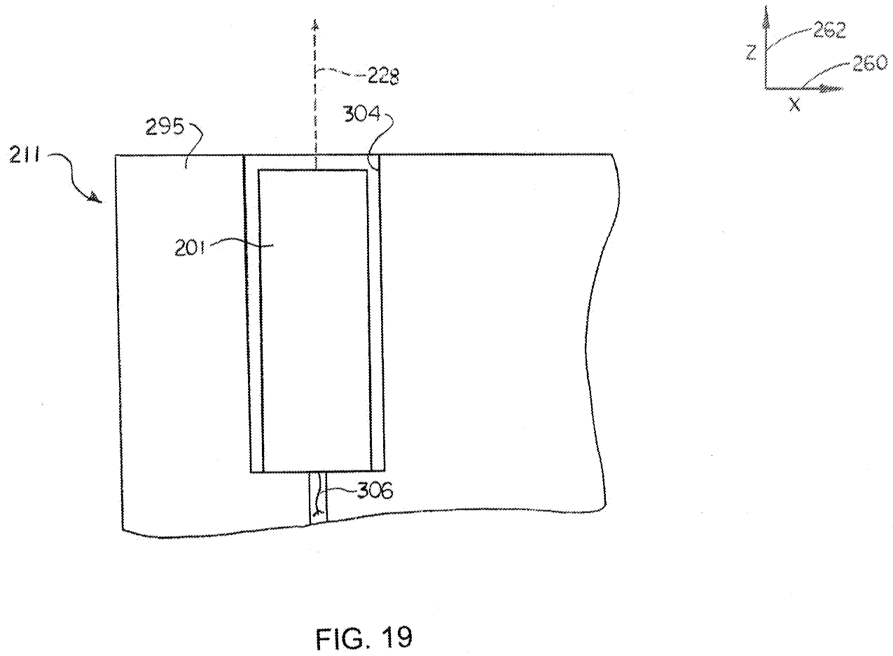

As illustrated in FIG. 19, in some embodiments, the horizontal equipment positioning light emitter 201 may be built into or manufactured in one piece with the main housing 295 or other component of the operating microscope 211. Accordingly, a light emitter cavity 304 may be provided in the main housing 295. The horizontal equipment positioning light emitter 201 may be mounted in the light emitter cavity 304 using suitable brackets and/or other fasteners (not illustrated).

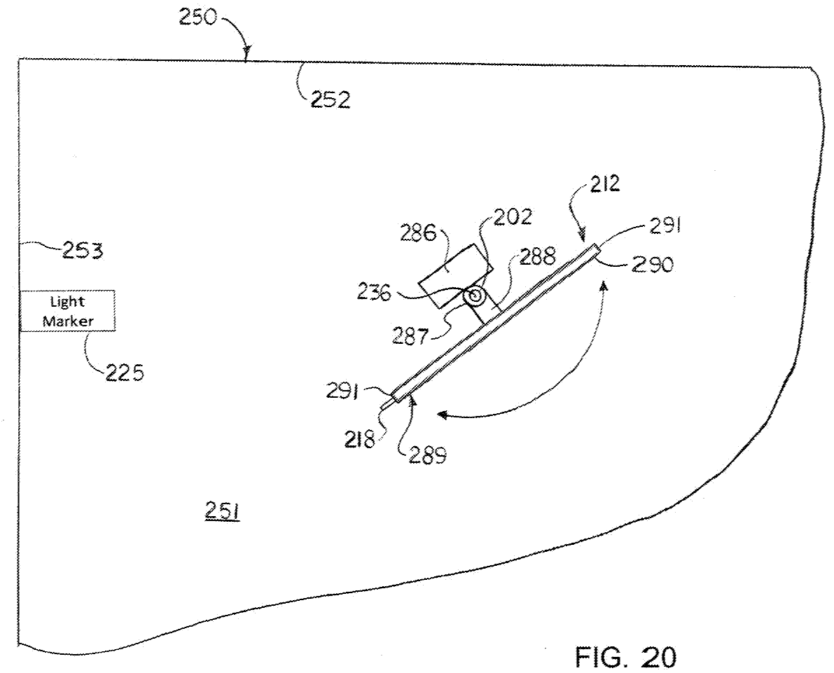

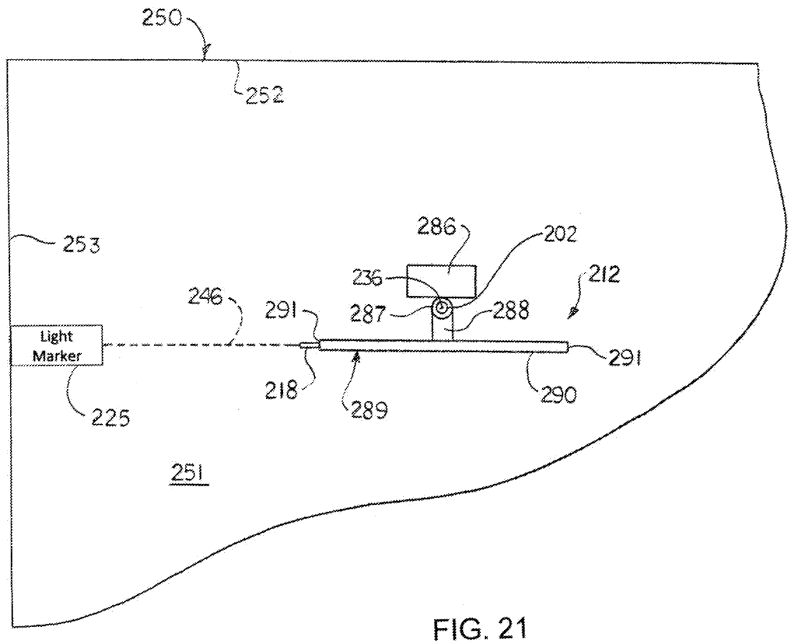

As illustrated in FIGS. 15, 20 and 21, some ophthalmic surgical procedures may utilize at least one visualization system 212. As illustrated in FIGS. 20 and 21, the visualization system 212 may include a typically wheeled base 286 which may support and render the visualization system 212 portable on the OR floor 251. A post 287 may extend upwardly from the base 286. A display screen 289 may be mounted to the post 287 via a screen support frame, bracket or arm 288. The display screen 289 may be adjustably rotatable about a rotational axis 236 defined typically by the longitudinal vertical axis of the post 287. For example and without limitation, in some embodiments, the visualization system 212 may include an NGENUITY® ophthalmic surgical visualization system known in the art. The rotational axis 236 may coincide with the vertical Z axis 262.

As illustrated in FIG. 15, the visualization system 212 may have a visualization system operating position 282 on the OR floor 251. In some applications, the visualization system operating position 282 may be generally on the side of the surgical patient table 216 which is opposite the operating microscope 211. A horizontal equipment positioning light emitter 202 may be provided on the visualization system 212. For example and without limitation, as illustrated in FIGS. 20 and 21, in some embodiments, the horizontal equipment positioning light emitter 202 may be provided on the post 287 of the visualization system 212 at the rotational axis 236 of the display screen 289. As illustrated in FIGS. 18 and 19, the horizontal equipment positioning light emitter 202 may be oriented upwardly to emit an upward light beam 228. As illustrated in FIG. 16, a second elevated light marker 222 may be provided on the OR ceiling 255 or other elevated structure in the operating room 250 directly above the visualization system operating position 282. Accordingly, when the visualization system 212 is in the visualization system operating position 282, as illustrated in FIG. 15, the upward light beam 228 emitted by the horizontal equipment positioning light emitter 202 may positionally coincide with and impinge on the second elevated light marker 222 at a second light beam impingement 232, as illustrated in FIG. 16.

As further illustrated in FIGS. 20 and 21, in some embodiments, the system 200 may include a rotational equipment positioning light emitter 218 which may be provided on the display screen 289 of the visualization system 212. The rotational equipment positioning light emitter 218 may be oriented horizontally to emit a typically horizontal side light beam 246 (FIG. 21) sideways from the display screen 289. A side light marker 225 may be provided on a side OR wall 253, as illustrated, or alternatively, on the rear OR wall 252 or the front OR wall 254 of the OR 250. The side light marker 225 may be positioned such that the side light beam 246 emitted by the rotational equipment positioning light emitter 218 impinges on the side light marker 225 when the display screen 289 of the visualization system 212 is oriented at the selected viewing angle or orientation, or horizontal rotational position, for optimum viewing by the surgeon 270 (FIG. 15) during the surgical procedure.

As illustrated in FIG. 15, some ophthalmic surgical procedures may utilize a phacoemulsification unit 213. The phacoemulsification unit 213 may be used to remove a cataract from the right eye 267 of the patient 266. The phacoemulsification unit 213 may be situated adjacent to the surgeon 270 for ease of access by the surgeon 270 during the surgical procedure.

The phacoemulsification unit 213 may have a standard or conventional design with a handpiece, a foot pedal, irrigation hardware and an aspiration system (not illustrated). A horizontal equipment positioning light emitter 203 may be provided on the phacoemulsification unit 213. The horizontal equipment positioning light emitter 203 may be oriented upwardly to emit an upward light beam 228.

As illustrated in FIG. 16, a third elevated light marker 223 may be provided on the OR ceiling 255 or other elevated structure in the operating room 250 directly above the phacoemulsification unit operating position 283. Accordingly, when the phacoemulsification unit 213 is in the phacoemulsification unit operating position 283, as illustrated in FIG. 15, the upward light beam 228 emitted by the horizontal equipment positioning light emitter 203 may positionally coincide with and impinge on the third elevated light marker 223 at a third light beam impingement 233, as illustrated in FIG. 16.

Operation of the system 200 may be as was heretofore described with respect to operation of the system 100. Accordingly, the horizontal equipment positioning light emitters 201, 202, 203 may each be operated to emit the corresponding upward light beam 228. The operation microscope 211, the visualization system 212 and the phacoemulsification unit 213 may be moved within the horizontal plane 264 defined by the X axis 260 and the Y axis 261 and properly placed at the respective microscope operating position 281, visualization system operating position 282 and phacoemulsification unit operating position 283 as indicated by impingement of each upward light beam 228 on its corresponding elevated light marker 221, 222, 223.

The surgical patient table 216 with the patient 266 thereon may be moved within the horizontal plane 264 until the downward light beam crosshairs 240 of the downward light beam 238, emitted by the horizontal patient positioning light emitter 210, impinge on the right eye 267 of the patient 266, thus indicating that the surgical patient table 216 is in the horizontal patient operating position 278. In some applications, the vertical patient positioning light emitter 208 may be operated to emit the angular light beam 242 and the surgical patient table 216 raised until the angular light beam crosshairs 244 of the angular light beam 242 impinge on the right eye 267 of the patient 266 and typically coincide with or intersect the downward light beam crosshairs 240 of the downward light beam 238, after which the surgical patient table 216 may be locked or secured at the selected vertical operating position.

In some applications, the rotational equipment positioning light emitter 218 may be operated to emit the side light beam 246 and the display screen 289 of the visualization system 212 rotated until the side light beam 246 impinges on the side light marker 225, as illustrated in FIG. 21, thus indicating that the display screen 289 is oriented at the optimum viewing angle for the surgeon 270. After patient and equipment positioning is completed, the horizontal equipment positioning light emitters 201, 202, 203, the horizontal patient positioning light emitter 210, the vertical patient positioning light emitter 208 and the rotational equipment positioning light emitter 218 may then be turned off and the surgical procedure commenced.

In some applications, the system 200 may be implemented in such a manner that the operating microscope 211 serves as an anchor point for subsequent positioning of the surgical patient table 116, the visualization system 212, the phacoemulsification unit 213 and/or other surgical equipment unit(s) in the operating room 250. For example and without limitation, the operating microscope 211 may initially be deployed in its corresponding equipment operating position 281 typically via impingement of the upward light beam 228, emitted from the horizontal equipment positioning light emitter 201, on the light marker 221 at the first light beam impingement 231. The operating microscope 211 would then be the anchor point for subsequent positioning of the surgical patient table 216, typically with the patient 266 resting thereon, at the patient operating position 278. The surgical patient table 216 may then be positioned relative to the operating microscope 211 at the patient operating position 278 by using the horizontal patient positioning light emitter 210, typically as was heretofore described, or by using any other suitable means, such as by estimating or otherwise determining the proper location of the patient operating position 278 and then rolling or moving the surgical patient table 216 into operating proximity to the operating microscope 211. The arm 296 and ocular head 297 of the operating microscope 211 may be positioned such that the ocular head 297 is positioned over the eye 267 of the patient 266. The visualization system 212, the phacoemulsification unit 213 and/or other surgical equipment unit(s) may then be positioned relative to the surgical patient table 216 and/or the operating microscope 211, typically using the respective light markers 222, 223 or by using any other suitable means, such as by estimating or otherwise determining the proper locations of the equipment operating positions 282, 283 relative to the operating microscope 211 and then rolling or moving the visualization system 212 and the phacoemulsification unit 213 on the OR floor 251 to the respective equipment operating positions 282, 283, for example and without limitation.

In some applications, the operating microscope 211 may be programmed or configured to automatically roll into a home position or baseline position which may correspond to its equipment operating position 281. Accordingly, the base 293 of the operating microscope 211 may be fitted with wheel motors (not illustrated) configured to rotate the wheels on the base 293. At least one controller (not illustrated) may operably interface with the wheel motors to facilitate proper positioning of the base 293 at the equipment operating position 281 responsive to rotation of the wheels on the OR floor 251. The controller may utilize a GPS and/or other guidance system to guide or control the direction and rotation of the wheels on the base 293 to facilitate precise positioning of the base 293 at the equipment operating position 281, typically according to the knowledge of those skilled in the art. The surgical patient table 216 may then be manually rolled on the OR floor 251 until the surgical patient table 216 is properly positioned at the patient operating position 278 and the patient 266 resting on the surgical patient table 216 is positioned beneath the ocular head 297 on the arm 296 of the operating microscope 211. The visualization system 212, the phacoemulsification unit 213 and/or other surgical equipment unit(s) may then be positioned relative to the surgical patient table 116 and/or the operating microscope 211, typically using the respective light markers 122, 123, 124 or using any other suitable means.

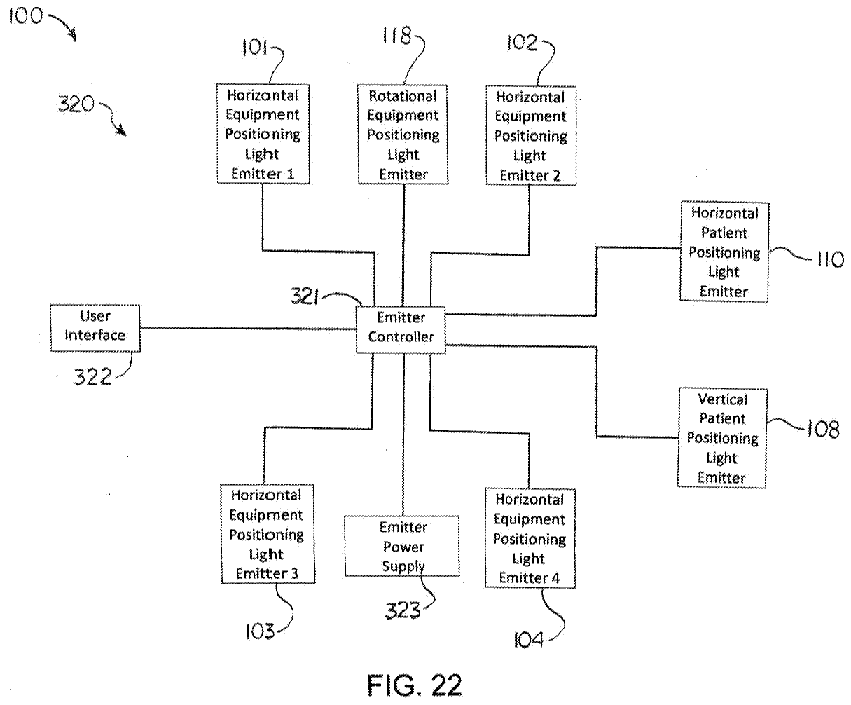

Referring next to FIG. 22 of the drawings, a typical control system 320 which is suitable for implementation of the system 100 or the system 200, hereinafter system 100, is illustrated. In the control system 320, a centralized user interface 322 may be configured to operate the horizontal equipment positioning light emitters 101, 102, 103, 104 as well as the horizontal patient positioning light emitter 110, the vertical patient positioning light emitter 108 and the rotational equipment positioning light emitter 118 of the system 100. Accordingly, an emitter controller 321 may operably interface with the user interface 322 and with the horizontal equipment positioning light emitters 101, 102, 103, 104, the horizontal patient positioning light emitter 110, the vertical patient positioning light emitter 108 and the rotational equipment positioning light emitter 118. The emitter controller 321 may be configured to facilitate control of the various components of the system 100 by user input via the user interface 322. At least one emitter power supply 323 may interface with the emitter controller 321. In some embodiments, the emitter power supply 323 may include a standard electrical outlet which may be on a wall, floor, ceiling, power module, or other surface or component in the operating room 150.

The user interface 322 of the control system 320 may include any control feature or combination of control features which may be suitable for controlling the upward light beams 128 of the horizontal equipment positioning light emitters 101, 102, 103, 104 and the side light beam 146 emitted by the rotational equipment positioning light emitter 118, as well as the downward light beam 138 emitted by the horizontal patient positioning emitter 110 and the angular light beam 142 emitted by the vertical patient positioning light emitter 108. For example and without limitation, suitable control features may include buttons, switches, dials, and touchscreen displays, or combinations thereof. The control feature or features of the user interface 322 may be configured to control the horizontal equipment positioning light emitters 101, 102, 103, 104, the rotational equipment positioning light emitter 118, the horizontal patient positioning emitter 110 and the vertical patient positioning light emitter 108 either individually or collectively.

The user interface 322 of the control system 320 may be provided in any location or position which is accessible by the surgeon 170, the surgical assistant or assistants 172 and/or other operating room personnel. For example and without limitation, in some embodiments, the user interface 322 may be configured as a mobile wireless remote control unit which enables the surgeon 170, the surgical assistant or assistants 172 or the other personnel to remotely control of the components of the system 100. The control system 320 with the user interface 322 may be configured as an app which can be operated on a smartphone, tablet computer or the like. In some embodiments, the user interface 322 may be provided as a control panel on the rear OR wall 152, one of the side OR walls 153, the front OR wall 154 and/or other surface, component or module in the operating room 150. For example and without limitation, in some embodiments, the user interface 322 may be provided on a portable control module which can be transported via rollers on the OR floor 151.

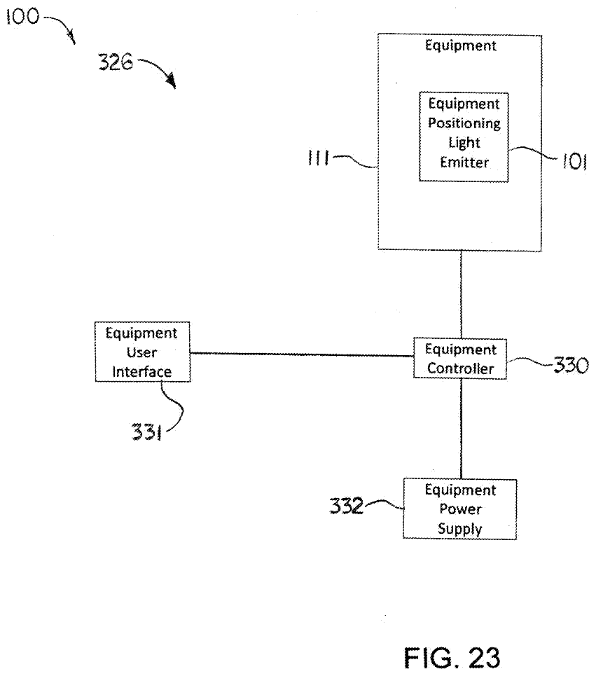

Referring next to FIG. 23 of the drawings, a typical alternative control system 326 which is suitable for implementation of the system 100, 200 is illustrated. In the control system 326, an equipment user interface 331 which controls each surgical equipment unit 111 may also be configured to operate the corresponding equipment positioning light emitter 101 on the surgical equipment unit 111. The equipment user interface 331 may interface with an equipment controller 330 which is configured to operate both the surgical equipment unit 111 and the equipment positioning light emitter 101 of the surgical equipment unit 111 via user manipulation of the equipment user interface 331. Accordingly, the equipment user interface 331 may include one or more control features which can be manipulated to control the equipment positioning light emitter 101. An equipment power supply 332 may electrically interface with the equipment controller 330. The equipment power supply 332 may provide a source of electrical power for both the surgical equipment unit 111 and the equipment positioning light emitter 101. In some embodiments, the equipment user interface 331 of the control system 326 may be configured to control the rotational equipment positioning light emitter 118 in the same or a similar manner.

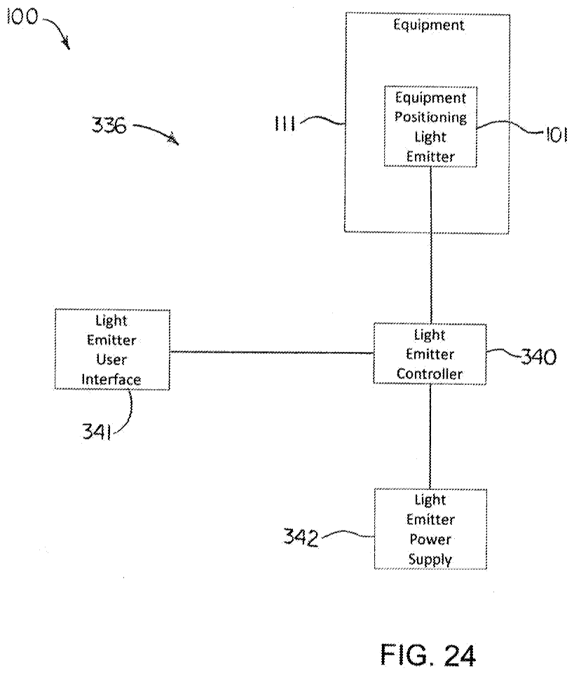

Referring next to FIG. 24 of the drawings, another alternative control system 336 in which the equipment positioning light emitter 101 on each surgical equipment unit 111 has its own dedicated light emitter user interface 341 and light emitter power supply 342 is illustrated. In some embodiments, a light emitter controller 340 may controllably interface with the equipment positioning light emitter 101. The light emitter user interface 341 and the light emitter power supply 342 may interface with the light emitter controller 340. The light emitter user interface 341 may be manipulated to facilitate control of the equipment positioning light emitter 101 via the light emitter controller 340. Alternatively, the light emitter controller 340 may be omitted and the light emitter power supply 342 may interface with the equipment positioning light emitter 101 through the light emitter user interface 341.

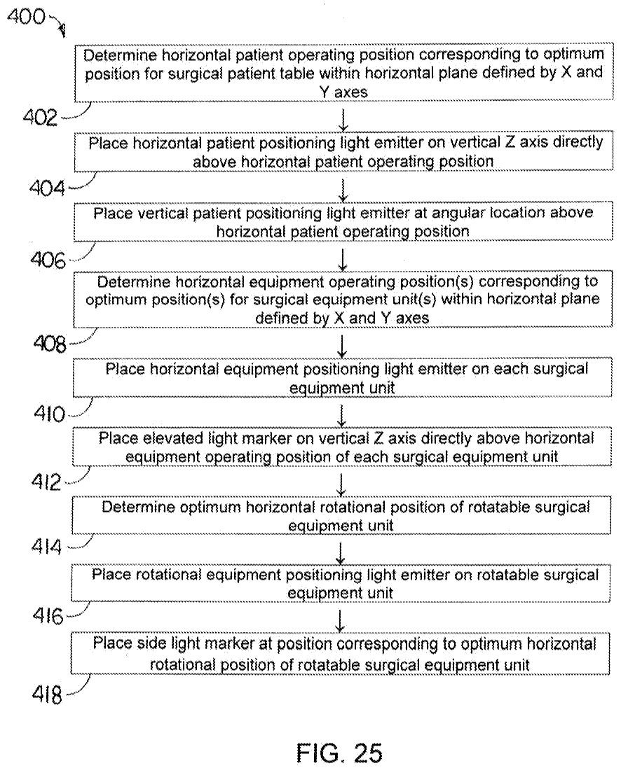

Referring next to FIG. 25 of the drawings, a flow diagram 400 which illustrates a typical method of assembling or setting up an illustrative embodiment of the surgical patient and/or equipment alignment systems 100, 200 is illustrated. At Step 402, a horizontal patient operating position which corresponds to an optimum position for a surgical patient table within a horizontal plane defined by X and Y axes on an OR floor in an operating room may be determined.

At Step 404, a horizontal patient positioning light emitter may be placed on a vertical Z axis directly above the horizontal patient operating position.

At Step 406, a vertical patient positioning light emitter may be placed at an angular location above the horizontal patient operating position.

At Step 408, at least one horizontal equipment operating position corresponding to the optimum position for at least one surgical equipment unit within the horizontal plane defined by the X and Y axes may be determined

At Step 410, a horizontal equipment positioning light emitter may be placed on each surgical equipment unit.

At Step 412, an elevated light marker may be placed on the vertical Z axis directly above the horizontal equipment operating position of each surgical equipment unit.

At Step 414, an optimum horizontal rotational position of at least one rotatable surgical equipment unit or a rotatable component of the rotatable surgical equipment unit may be determined.

At Step 416, a rotational equipment positioning light emitter may be placed on each rotatable surgical equipment unit or the rotatable component thereof.

At Step 418, a side light marker may be placed at a position which corresponds to the optimum horizontal rotational position of each rotatable surgical equipment unit or the rotatable component.

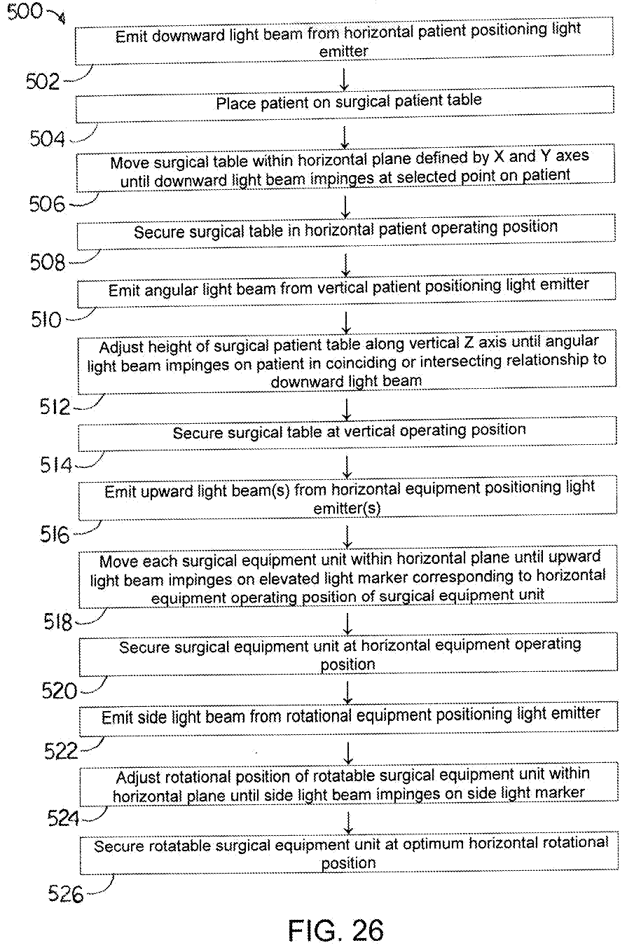

Referring next to FIG. 26 of the drawings, a flow diagram 500 of an illustrative embodiment of the surgical patient and/or equipment alignment methods of the disclosure is illustrated. At Step 502, a downward light beam may be emitted from the horizontal patient positioning light emitter.

At Step 504, the patient may be placed on the surgical patient table.

At Step 506, the surgical patient table may be moved within the horizontal plane defined by the X and Y axes until the downward light beam impinges at a selected point on the patient. In some applications, the selected point may be a surgical site.

At Step 508, the surgical patient table may be secured in the horizontal patient operating position.

At Step 510, an angular light beam may be emitted from the vertical patient positioning light emitter.

At Step 512, the height of the surgical patient table may be adjusted along the vertical Z axis until the angular light beam impinges on the patient typically in coinciding or intersecting relationship to the downward light beam.

At Step 514, the surgical patient table may be secured at the vertical operating position.

At Step 516, an upward light beam may be emitted from each horizontal equipment positioning light emitter.

At Step 518, each surgical equipment unit may be moved within the horizontal plane until the upward light beam impinges on the elevated light marker which corresponds to the horizontal equipment operating position of the surgical equipment unit.

At Step 520, the surgical equipment unit may be locked or secured at the horizontal equipment operating position.

At Step 522, a side light beam may be emitted from the rotational equipment positioning light emitter.

At Step 524, the rotational position of the rotatable surgical equipment unit or the rotatable component thereof may be adjusted within the horizontal plane until the side light beam impinges on the side light marker.

At Step 526, the rotatable surgical equipment unit or the rotatable component thereof may be locked or secured at the optimum horizontal rotational position.

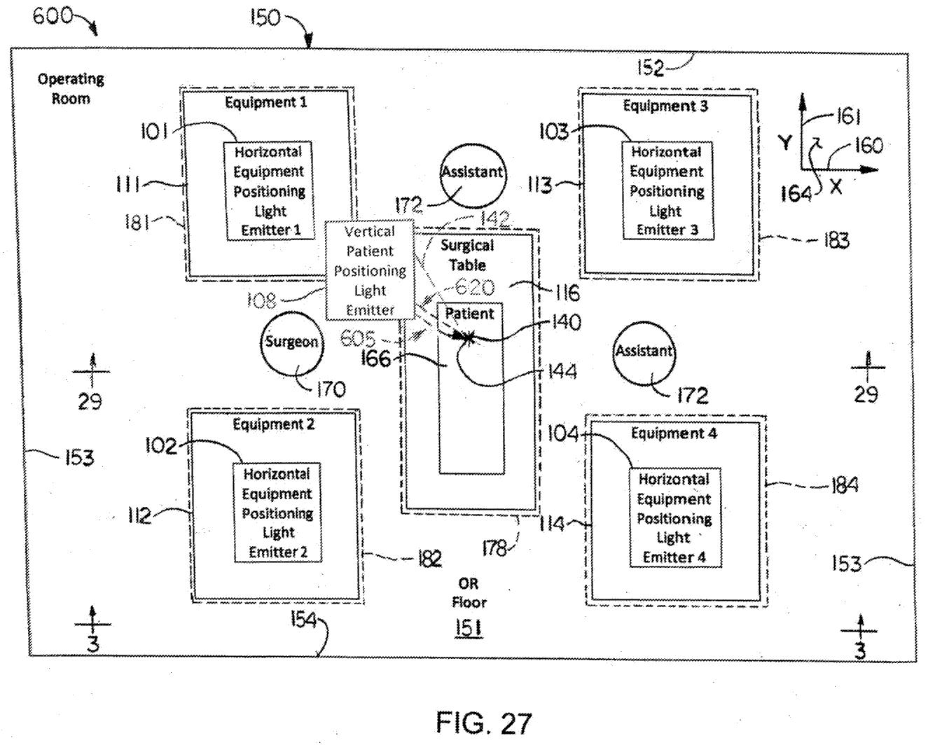

Referring next to FIGS. 27-30 of the drawings, an alternative illustrative embodiment of the surgical patient and/or equipment alignment systems of the disclosure is generally indicated by reference number 600. In the system 600, components which are structurally and/or functionally analogous to the components of the system 100 heretofore described with respect to FIGS. 1-14 are indicated by the same respective reference numbers. Accordingly, the same description which was heretofore set forth with respect to the system 100 is incorporated by reference herein in its entirety with respect to the system 600.

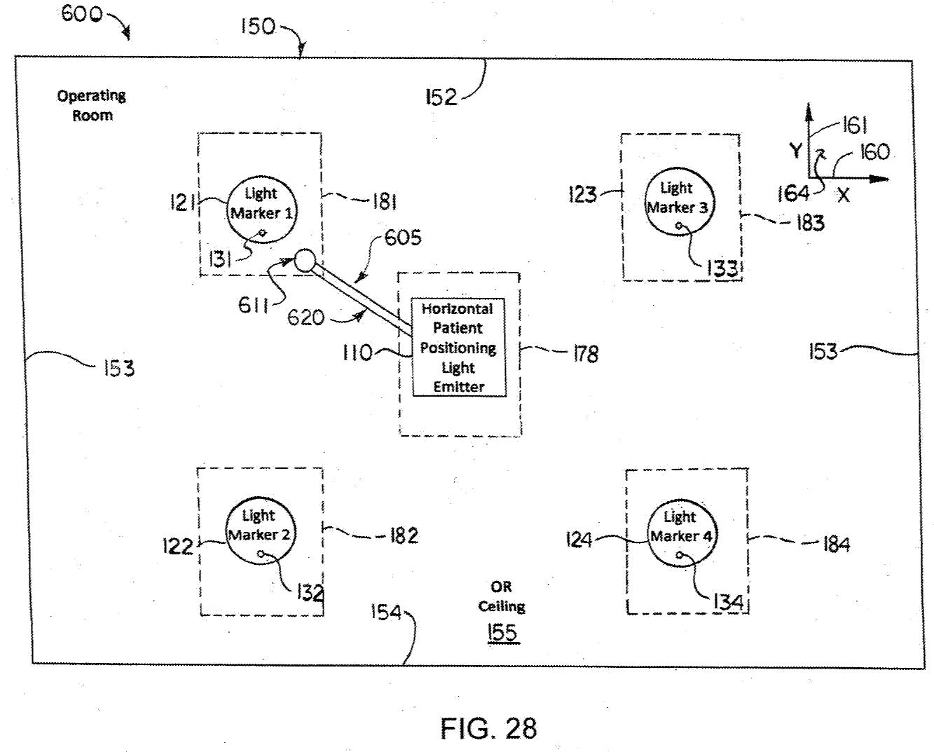

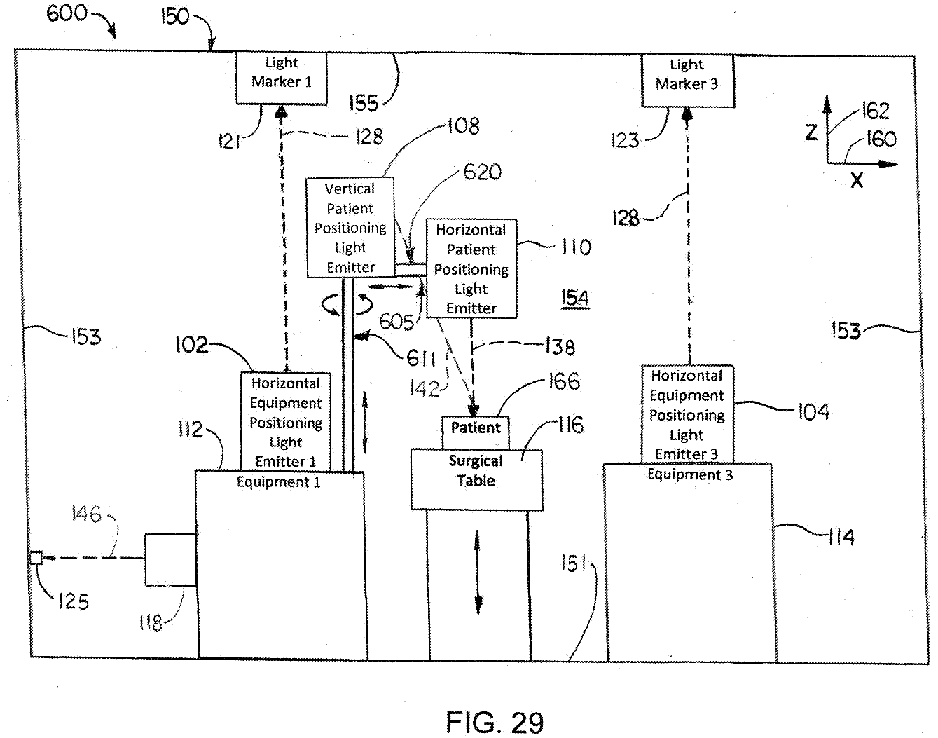

In the system 600, an emitter positioning assembly 605 (illustrated in phantom in FIG. 27) may be supported by a surgical equipment unit 111, 112, 113, 114, such as the first surgical equipment unit 111, as illustrated, for example and without limitation. The horizontal patient positioning light emitter 110 (FIGS. 28 and 29) may be supported by the emitter positioning assembly 605. Accordingly, in typical implementation of the system 600, which will be hereinafter described, the surgical equipment unit 111 may initially be deployed in its optimum equipment operating position 181 in the operating room 150, typically in the manner which was heretofore described with respect to the system 100. The emitter positioning assembly 605 may be configured to position the horizontal patient positioning light emitter 110 over the OR floor 151 such that the surgical patient table 116 with the patient 166 typically reclining thereon may then be moved into the optimum patient operating position 178 on the OR floor 151 as indicated by impingement of the downward light beam 138 (FIG. 29) emitted by the horizontal patient positioning light emitter 110 on the patient 166. The initially properly positioned surgical equipment unit 111 may thus serve as a guide or template for subsequent proper positioning of the surgical patient table 116 and the patient 166 in the optimum patient operating position 178 within the horizontal plane 164 (FIG. 27) formed by the X axis 160 and the Y axis 161 for the surgical procedure.

In some embodiments of the system 600, the vertical patient positioning light emitter 108 may additionally or alternatively be supported by the emitter positioning assembly 605. The emitter positioning assembly 605 may be configured to support the vertical patient positioning light emitter 108 in such a manner and position that the surgical patient table 116 and the patient 166 may be adjusted to the optimum vertical patient operating position as indicated by impingement of the angular light beam 142 emitted by the vertical patient positioning light emitter 108 on the patient 166 typically in coinciding relationship to the downward light beam 138 at the downward light beam crosshairs 140. The surgical equipment unit 111 may thus additionally or alternatively serve as a guide or template for subsequent proper vertical positioning of the surgical patient table 116 and the patient 166 along the vertical Z axis 162 (FIG. 29) for the surgical procedure.

In some embodiments, the emitter positioning assembly 605 may include a vertical assembly arm 611. The vertical assembly arm 611 may be configured to extend along the Z axis 162 (FIG. 29) in the operating room 150. A horizontal assembly arm 620 may extend from the vertical assembly arm 611. The horizontal assembly arm 620 may be configured to extend along a horizontal axis 160 in the operating room 150. The horizontal patient positioning light emitter 110 may be supported by the horizontal assembly arm 620. The vertical patient positioning light emitter 110 may be supported by the vertical assembly arm 611 and/or the horizontal assembly arm 620.

In some embodiments, the vertical assembly arm 611 of the emitter positioning assembly 605 may be selectively vertically adjustable along the Z axis 162 typically according to the knowledge of those skilled in the art. Likewise, the horizontal assembly arm 620 may be selectively horizontally adjustable along the horizontal axis 160. In some embodiments, the vertical assembly arm 611 may be rotatably adjustable with respect to the surgical equipment unit 111 and about the Z axis 162 typically according to the knowledge of those skilled in the art.

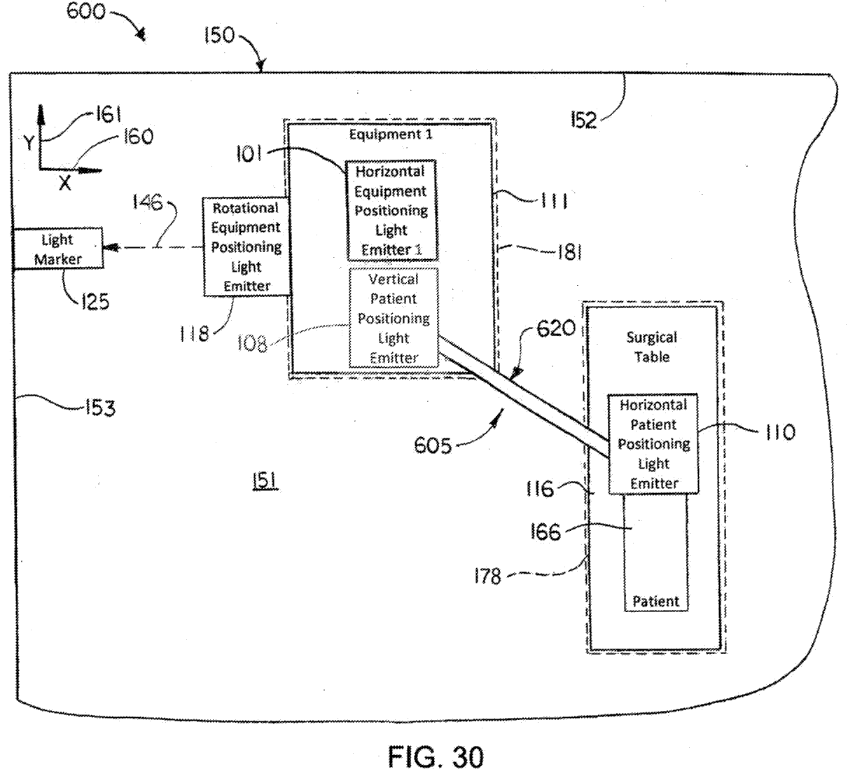

In typical application of the system 600, the surgical equipment units 111, 112, 113, 114 may be moved into their respective operating positions 181, 182, 183, 184. In some applications, this may be accomplished with respect to the surgical equipment unit 111, for example, by adjusting the position of the surgical equipment unit 111 along the X axis 160 and the Y axis 161 within the horizontal plane 164 via impingement of the upward light beam 128 emitted from the horizontal equipment positioning light emitter 102 onto the elevated light marker 121 at the light beam impingement 131, as illustrated in FIGS. 28 and 29. In some applications, the position of the surgical equipment unit 111 may be rotationally adjusted within the horizontal plane 164 by impingement of the side light beam 146 emitted from the rotational equipment positioning light emitter 118 onto the side light marker 125, as illustrated in FIGS. 29 and 30. Accordingly, the surgical equipment unit 111, now deployed within its optimum equipment operating position 181, may thus serve as a guide, template or anchor point for subsequent proper positioning of the surgical patient table 116 and the patient 166 in the optimum patient operating position 178 within the horizontal plane 164 and/or along the Z axis 162 for the surgical procedure, as will be hereinafter described.

After the surgical equipment unit 111 is properly positioned within its optimum equipment operating position 181, the downward light beam 138 may be emitted from the horizontal patient positioning light emitter 110 on the horizontal assembly arm 620 of the emitter positioning assembly 605. The downward light beam 138 may initially impinge on the OR floor 151 of the operating room 150. The surgical patient table 116 with the patient 166 reclining thereon may then be moved within the horizontal plane 164 until the downward light beam 138 impinges on the patient 166, thereby forming the downward light beam crosshairs 140 (FIG. 27) at the surgical site on the patient 166 and indicating that the surgical patient table 116 is at the optimum patient operating position 178. As illustrated in FIGS. 27 and 29, in some applications, the vertical patient positioning light emitter 108 may be operated to emit the angular light beam 142. The vertical position of the surgical patient table 116 may be adjusted until the angular light beam 142 impinges on the patient 166 and forms the angular light beam crosshairs 144 which may coincide with the downward light beam crosshairs 140 of the downward light beam 138 to mark the surgical site on the patient 166, typically as was heretofore described.

Prior to positioning of the patient 166 for the surgical procedure, as was heretofore described, the proper rotational orientation or position of the vertical assembly arm 611, and thus, the position of the horizontal assembly arm 620 within the horizontal plane 164, as well as the length of the horizontal assembly arm 620 of the emitter positioning assembly 605, may be determined to obtain the correct position of the horizontal patient positioning light emitter 110 for proper positioning of the surgical patient table 116 and the patient 166 at the optimum patient operating position 178 for a given surgical procedure. In some applications, these determinations may be made by trial and error. The position and length of the horizontal assembly arm 620 for deployment of the horizontal patient positioning light emitter 110 in the proper position within the horizontal plane 164 may vary depending on the particular type of surgical equipment 111 which supports the emitter positioning assembly 605, as well as the type or nature of the surgical procedure which is carried out.

In some embodiments, the vertical patient positioning light emitter 108 may be mounted on the emitter positioning assembly 605 according to the knowledge of those skilled in the art. For example and without limitation, in some embodiments, a mount bracket, arm, clip, clamp, platform and/or other light emitter support (not illustrated) may mount the vertical patient positioning light emitter 108 to the vertical assembly arm 611, the horizontal assembly arm 620 and/or other structural component or components of the emitter positioning assembly 605. The light emitter support may be configured to facilitate rotational or pivotal adjustment of the vertical patient positioning light emitter 108 within the horizontal plane 164 to facilitate accurate aiming of the angular light beam 142 onto the surgical site of the patient 166. In some embodiments, the emitter positioning assembly 605 may additionally or alternatively facilitate vertical positioning of the vertical patient positioning light emitter 108 along the vertical Z axis 162 typically according to the knowledge of those skilled in the art.

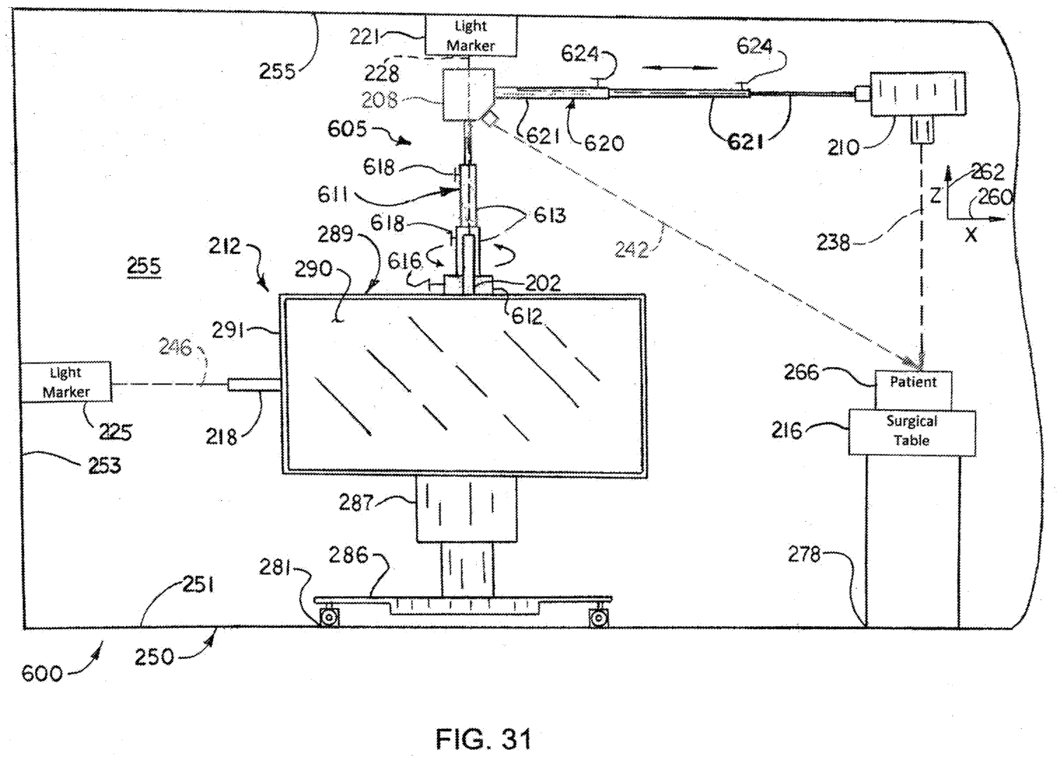

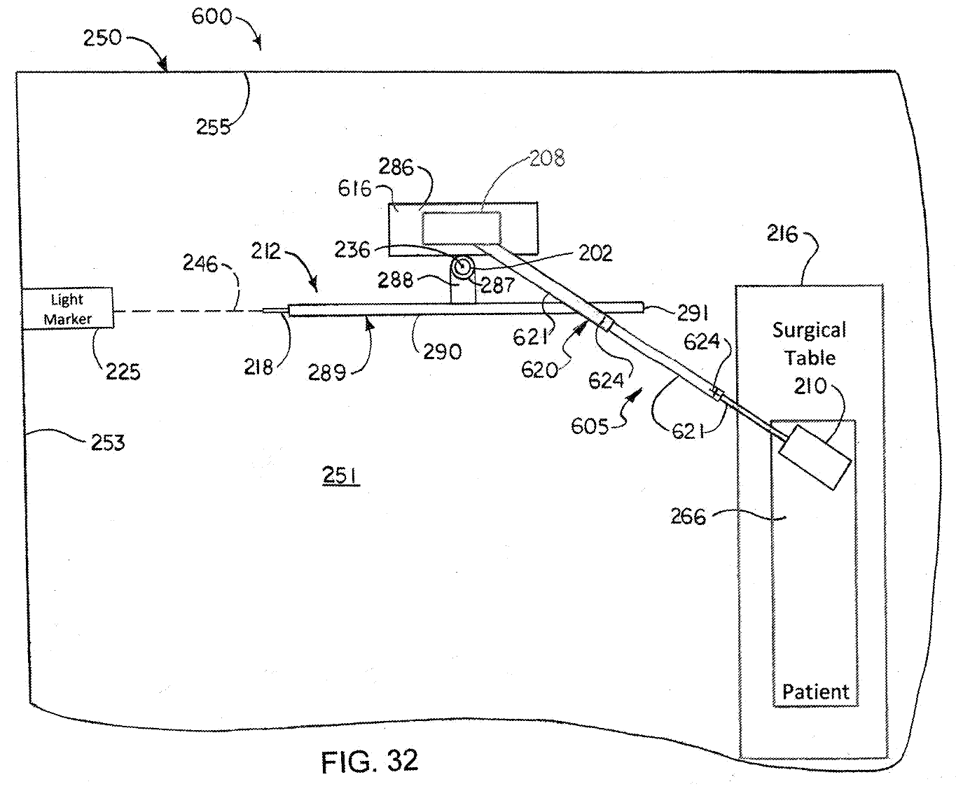

Referring next to FIGS. 31 and 32 of the drawings, an exemplary embodiment of the system 600 in which the emitter positioning assembly 605 is supported by a visualization system 212 is illustrated. In the system 600 illustrated in FIGS. 31-33, the visualization system 212 may have the same or similar description to that of the visualization system 212 heretofore described with respect to FIGS. 20 and 21, with like components designated by like reference numbers. Thus, to the extent which is applicable, the same description which was heretofore described with respect to the system 200 is incorporated by reference herein in its entirety herein with respect to the system 600.

The vertical assembly arm 611 of the emitter positioning assembly 605 may be supported by the base 286, the post 287 (FIG. 32) and/or other structural component(s) of the visualization system 212. In some embodiments, the vertical assembly arm 611 may be rotatable about a vertical Z axis 262 (FIG. 31). Accordingly, an arm base 612 may be provided on the visualization system 212. The vertical assembly arm 611 may be rotatably mounted in the arm base 612. A rotational adjustment control 616 may operably engage the vertical assembly arm 611. The rotational adjustment control 616 may be configured to lock the vertical assembly arm 611 at a desired 360-degree rotational position along the vertical Z axis 262. For example and without limitation, in some embodiments, the rotational adjustment control 616 may include a threaded knob which extends through a threaded opening (not illustrated) in the arm base 612 and engages the vertical assembly arm 611. Alternatively, the rotational adjustment control 616 may include a pin (not illustrated) which extends through a pin opening (not illustrated) in the arm base 612 and engages a registering detent or pin opening (not illustrated) in the vertical assembly arm 611 to lock the vertical assembly arm 611 at the desired rotational position. In some embodiments, the rotational adjustment control 616 may be electric, pneumatic, or hydraulic.

In some embodiments, the vertical assembly arm 611 may have a telescoping construction. Accordingly, multiple telescoping vertical arm segments 613 may extend from the arm base 612 and from each other. At least one vertical adjustment control 618 may facilitate securement of the telescoping vertical arm segments 613 to secure the vertical assembly arm 611 at the selected height. For example and without limitation, in some embodiments, each vertical adjustment control 618 may include a threaded knob which extends through a threaded opening (not illustrated) in each vertical arm segment 613 and engages the corresponding telescopically extending vertical arm segment 613. Alternatively, each vertical adjustment control 618 may include a pin (not illustrated) which extends through a pin opening (not illustrated) in each corresponding vertical arm segment 613 and engages a selected registering one of multiple detents or pin openings (not illustrated) in the corresponding telescoping extending vertical arm segment 613. In some embodiments, the vertical adjustment control 618 may be electric, pneumatic, or hydraulic.

In some embodiments, the horizontal assembly arm 620 may have a telescoping construction. Accordingly, multiple telescoping horizontal arm segments 621 may extend from the terminal vertical arm segment 613 of the vertical assembly arm 611 and from each other. At least one horizontal adjustment control 624 may facilitate securement of the telescoping horizontal arm segments 621 to secure the horizontal assembly arm 620 at the selected length. For example and without limitation, in some embodiments, each horizontal adjustment control 624 may include a threaded knob which extends through a threaded opening (not illustrated) in each corresponding horizontal arm segment 621 and engages the corresponding telescopically extending horizontal arm segment 621. Alternatively, each horizontal adjustment control 624 may include a pin (not illustrated) which extends through a pin opening (not illustrated) in each corresponding horizontal arm segment 621 and engages a selected registering one of multiple detents or pin openings (not illustrated) in the corresponding telescoping extending horizontal arm segment 621. In some embodiments, the horizontal adjustment control 624 may be electric, pneumatic, or hydraulic. The horizontal patient positioning light emitter 210 may be mounted on the terminal, distal or extending segment of the horizontal assembly arm segments 621, as illustrated. The vertical patient positioning light emitter 208 may be supported by the vertical assembly arm 611, as illustrated, the arm base 612, and/or other structural component or components of the emitter positioning assembly 605.

Application of the system 600 may be as was heretofore described with respect to the system 600 in FIGS. 27-30. Prior to the surgical procedure, the rotational position of the horizontal assembly arm 620 of the emitter positioning assembly 605 may be adjusted and locked by engagement of the rotational adjustment control 616. The selected length of the vertical assembly arm 611 may be secured by engagement of the vertical adjustment control or controls 618. The selected length of the horizontal assembly arm 620 may be secured by engagement of the horizontal adjustment control or controls 624. During the surgical procedure, the visualization system 212 may be operated as was heretofore described with respect to FIGS. 20 and 21.

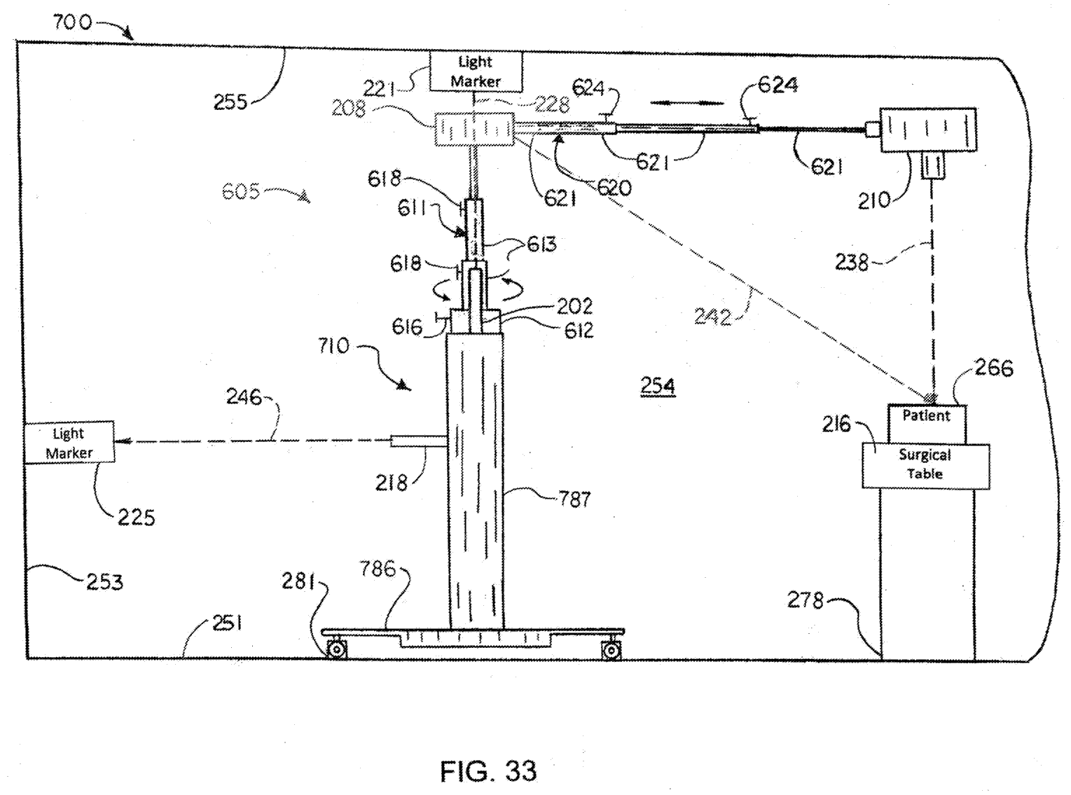

Referring next to FIG. 33 of the drawings, another alternative illustrative embodiment of the surgical patient and/or equipment alignment systems of the disclosure is generally indicated by reference number 700. Unless otherwise noted herein, the system 700 may have the same or similar description to that of the system 600 heretofore described with respect to FIGS. 31 and 32, with structurally and/or functionally analogous components designated by like reference numbers. Accordingly, to the extent applicable, the same description which was heretofore set forth with respect to the system 600 in FIGS. 31 and 32 is incorporated by reference herein in its entirety with respect to the system 700.

In the system 700, the emitter positioning assembly 605 may be mounted on an emitter assembly stand 710. The emitter assembly stand 710 may include a wheeled stand base 786. The wheels on the stand base 786 may include a wheel locking mechanism (not illustrated) which facilitates locking of the stand base 786 in place at the desired equipment operating position on the OR floor 251, typically in the conventional manner. A stand frame or post, hereinafter stand frame 787, may extend upwardly from the stand base 786. The emitter positioning assembly 605 may be supported by the stand frame 787.

In some embodiments, the horizontal equipment positioning light emitter 202 may be supported by the stand frame 787 in an upward emitting position. The rotational equipment position light emitter 218 may be supported by the stand frame 787 in a horizontal emitting position, typically according to the knowledge of those skilled in the art. The horizontal patient positioning light emitter 210 may be mounted on the terminal, distal or extending segment of the horizontal assembly arm segments 621, as illustrated. The vertical patient positioning light emitter 208 may be supported by the vertical assembly arm 611, as illustrated, the arm base 612, the stand base 786, and/or other structural component or components of the emitter assembly stand 710.

Application of the system 700 may be similar to that which was heretofore described with respect to the system 600 in FIGS. 31 and 32. Prior to the surgical procedure, the emitter assembly stand 710 may be maneuvered into its desired optimum horizontal equipment operating position 181 (FIG. 27) by emitting the upward light beam 228 from the horizontal equipment positioning light emitter 202 and rolling the emitter assembly stand 710 on the OR floor 251 until the upward light beam 228 impinges on the elevated light marker 221. The assembly stand 710 may be oriented in the desired rotational position by emitting the horizontal light beam 246 from the rotational equipment position light emitter 218 and rotating the emitter assembly stand 710 on the OR floor 251 until the horizontal light beam 246 impinges on the side light marker 225. The assembly stand 710 may then be locked in place to prevent inadvertent movement of the assembly stand 710 on the OR floor 251. Accordingly, the emitter assembly stand 710 may become the anchor point for subsequent positioning of the surgical patient table 216 and/or surgical equipment unit(s) 211, 212, 213, 214. The downward light beam 238 may then be emitted from the horizontal patient positioning light emitter 210 and the surgical patient table 216 with the patient 266 thereon moved into place until the downward light beam 238 impinges on the patient 266 to indicate that the surgical patient table 216 is at the optimum patient operating position 278, typically as was heretofore described with respect to the system 600. In some applications, the vertical patient positioning light emitter 208 may be operated to emit the angular light beam 242. The vertical position of the surgical patient table 216 may be adjusted until the angular light beam 242 impinges on the patient 266 and forms the angular light beam crosshairs 144 (FIG. 27) which may coincide with the downward light beam crosshairs 140 of the downward light beam 238 to mark the surgical site on the patient 266, typically as was heretofore described.

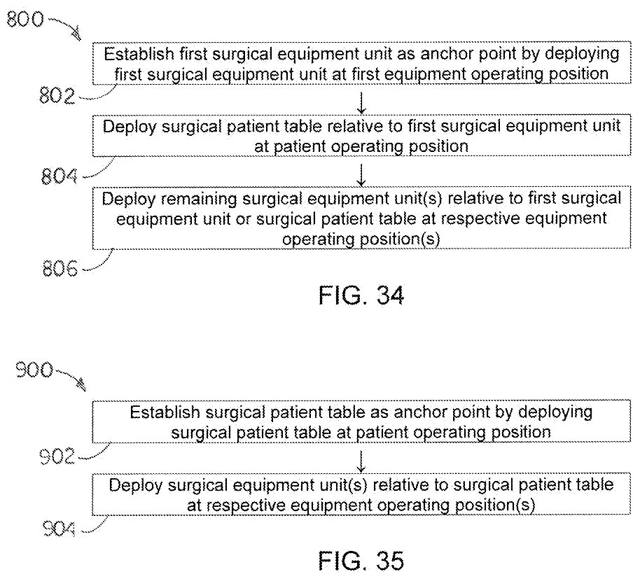

Referring next to FIG. 34 of the drawings, a flow diagram 800 of another illustrative embodiment of the surgical patient and/or equipment alignment methods of the disclosure in which a surgical equipment unit serves as an anchor point for positioning of the surgical patient table and/or the remaining surgical equipment unit(s) in the operating room is illustrated. At Step 802, a first surgical equipment unit may be established as an anchor point for positioning of the surgical patient table and/or the remaining surgical equipment units by deploying first surgical equipment unit at a first equipment operating position on the OR floor in an operating room.

At Step 804, the surgical patient table may be deployed relative to the first surgical equipment unit at a patient operating position on the OR floor.

At Step 806, the remaining surgical equipment unit(s) may be deployed relative to the first surgical equipment unit or the surgical patient table at the respective equipment operating position(s) on the OR floor.

Referring next to FIG. 35 of the drawings, a flow diagram 900 of another illustrative embodiment of the surgical patient and/or equipment alignment methods of the disclosure in which the surgical patient table serves as an anchor point for positioning of the surgical equipment unit(s) in the operating room is illustrated. At Step 902, a surgical patient table may be established as an anchor point by deploying the surgical patient table at a patient operating position on an OR floor in an operating room.

At Step 904, the remaining surgical equipment unit(s) may be deployed relative to the surgical patient table at the respective equipment operating position(s) on the OR floor.

While certain illustrative embodiments of the disclosure have been described above, it will be recognized and understood that various modifications can be made to the embodiments and the appended claims are intended to cover all such modifications which may fall within the spirit and scope of the disclosure.

I claim:

1. A surgical patient and/or equipment alignment system suitable for establishing as an anchor point and/or facilitating optimal positioning of a patient and/or at least one surgical equipment unit for a surgical procedure in an operating room, the system comprising at least one of the following:
a horizontal patient operating position corresponding to an optimal position of a surgical patient table configured to support the patient in the surgical procedure and a horizontal patient positioning light emitter directly above the horizontal patient operating position, the horizontal patient positioning light emitter configured to emit a downward light beam to impinge at a selected point on the patient when the surgical patient table is deployed in place at the horizontal patient operating position; and
at least one horizontal equipment operating position corresponding to an optimal position of the at least one surgical equipment unit, respectively, in the surgical procedure, at least one horizontal equipment positioning light emitter configured for placement on the at least one surgical equipment unit, respectively, and at least one elevated light marker directly above the at least one horizontal equipment operating position, respectively, the at least one horizontal equipment positioning light emitter configured to emit at least one upward light beam to impinge on the at least one elevated light marker at at least one light beam impingement, respectively, when the at least one surgical equipment unit is at the at least one horizontal equipment operating position, respectively.

2. The system of claim 1 wherein the at least one elevated light marker is an ink, paint or chalk spot or mark; a protrusion; an indentation; or a button, tab, disk, strip, or tape.

3. The system of claim 1 wherein the horizontal patient positioning light emitter comprises at least one optical projector.

4. The system of claim 1 wherein the downward light beam is configured to form downward light beam crosshairs at the selected point on the patient.

5. The system of claim 1 wherein the at least one upward light beam comprises at least one collimated laser beam.

6. The system of claim 1 further comprising at least one vertical patient positioning light emitter disposed in elevated angular relationship to the horizontal patient operating position, the at least one vertical patient positioning light emitter configured to emit at least one angular light beam, respectively, to facilitate the vertical positioning of the surgical patient table.

7. The system of claim 1 further comprising at least one side light marker and at least one rotational equipment positioning light emitter configured for placement on the at least one surgical equipment unit in spaced-apart relationship to the at least one side light marker, the at least one rotational equipment positioning light emitter configured to emit at least one side light beam for impingement on the at least one side light marker, respectively, to facilitate horizontal rotational positioning of the at least one surgical equipment unit within the at least one horizontal equipment operating position.

8. The system of claim 1 wherein the at least one horizontal equipment positioning light emitter is configured to be retrofitted to the at least one surgical equipment unit.

9. The system of claim 8 further comprising at least one light emitter mount assembly configured to mount the at least one horizontal equipment positioning light emitter on the at least one surgical equipment unit.

10. The system of claim 9 wherein the at least one light emitter mount assembly comprises at least one mount bracket configured for mounting on the at least one surgical equipment unit, and the at least one horizontal equipment positioning light emitter is supported by the at least one mount bracket.

11. The system of claim 1 wherein the at least one horizontal equipment positioning light emitter is configured to be built or manufactured in one piece with the at least one surgical equipment unit.

12. The system of claim 11 wherein the at least one horizontal equipment positioning light emitter is configured to be mounted in a light emitter cavity in the at least one surgical equipment unit.

13. The system of claim 1 further comprising an emitter positioning assembly configured to be carried by the at least one surgical equipment unit, and wherein the horizontal patient positioning light emitter is carried by the emitter positioning assembly.

14. The system of claim 13 wherein the emitter positioning assembly comprises a vertical assembly arm configured for mounting on the at least one surgical equipment unit and a horizontal assembly arm carried by the vertical assembly arm, and wherein the horizontal patient positioning light emitter is carried by the horizontal assembly arm.

15. A surgical patient and/or equipment alignment system suitable for establishing as an anchor point and/or facilitating optimal positioning of a patient and a plurality of surgical equipment units for a surgical procedure in an operating room having an operating room floor and an operating room ceiling, the system comprising:
a horizontal patient operating position for designation on the operating room floor, the horizontal patient operating position corresponding to an optimal position of a surgical patient table configured to support the patient in the surgical procedure;
a horizontal patient positioning light emitter for deployment on the operating room ceiling directly above the horizontal patient operating position, the horizontal patient positioning light emitter configured to emit a downward light beam to impinge at a selected point on the patient when the surgical patient table is at the horizontal patient operating position;
a plurality of horizontal equipment operating positions for location on the operating room floor, the plurality of horizontal equipment operating positions corresponding to optimal positions of the plurality of surgical equipment units, respectively, in the surgical procedure;
a plurality of horizontal equipment positioning light emitters configured for placement on the plurality of surgical equipment units, respectively;
a plurality of elevated light markers for deployment on the operating room ceiling directly above the plurality of horizontal equipment operating positions, respectively, the plurality of horizontal equipment positioning light emitters configured to emit a plurality of upward light beams to impinge on the plurality of elevated light markers at a plurality of light beam impingements, respectively, when the plurality of surgical equipment units are at the plurality of horizontal equipment operating positions, respectively; and
at least one vertical patient positioning light emitter for deployment in elevated angular relationship to the horizontal patient operating position, the at least one vertical patient positioning light emitter configured to emit at least one angular light beam, respectively, to facilitate vertical positioning of the surgical patient table.

16. The system of claim 15 wherein each of the plurality of elevated light markers is an ink, paint or chalk spot or mark; a protrusion; an indentation; or a button, tab, disk, strip, or tape.

17. The system of claim 15 wherein the horizontal patient positioning light emitter comprises at least one optical projector.

18. The system of claim 15 wherein the downward light beam is configured to form downward light beam crosshairs at the selected point on the patient.

19. The system of claim 15 wherein each of the plurality of upward light beams comprises a collimated laser beam.

20. The system of claim 15 further comprising at least one side light marker and at least one rotational equipment positioning light emitter configured for placement on at least one of the plurality of surgical equipment units, the at least one rotational equipment positioning light emitter configured to emit at least one side light beam for impingement on the at least one side light marker, respectively, to facilitate the horizontal rotational positioning of the at least one of the plurality of surgical equipment units within a corresponding one of the plurality of horizontal equipment operating positions.

21. The system of claim 15 wherein at least one of the plurality of horizontal equipment positioning light emitters is configured to be retrofitted to a corresponding one of the plurality of surgical equipment units.

22. The system of claim 21 further comprising at least one light emitter mount assembly configured to mount the at least one of the plurality of horizontal equipment positioning light emitters on the corresponding one of the plurality of surgical equipment units.

23. The system of claim 15 wherein at least one of the plurality of horizontal equipment positioning light emitters is configured to be built or manufactured in one piece with a corresponding one of the plurality of surgical equipment units.

24. The system of claim 23 wherein the at least one of the plurality of horizontal equipment positioning light emitters is configured to be mounted in a light emitter cavity in the corresponding one of the plurality of surgical equipment units.

25. The system of claim 15 further comprising an emitter positioning assembly configured to be carried by at least one of the plurality of surgical equipment units, and wherein the horizontal patient positioning light emitter is carried by the emitter positioning assembly.

26. The system of claim 25 wherein the emitter positioning assembly comprises a vertical assembly arm configured for mounting on the at least one of the plurality of surgical equipment units and a horizontal assembly arm carried by the vertical assembly arm, and wherein the horizontal patient positioning light emitter is carried by the horizontal assembly arm.

27. A surgical patient and/or equipment alignment method for optimal positioning of a patient and/or at least one surgical equipment unit for a surgical procedure in an operating room, the method comprising at least one of the following:
determining a horizontal patient operating position corresponding to an optimum position for a surgical patient table within a horizontal plane, emitting a downward light beam from a horizontal patient positioning light emitter directly above the horizontal patient operating position, placing the patent on a surgical patient table, moving the surgical patient table to the horizontal patient operating position by moving the surgical patient table within the horizontal plane until the downward light beam impinges at a selected point on the patient, and securing the surgical patient table at the horizontal patient operating position; and
determining a horizontal equipment operating position corresponding to an optimum position for the at least one surgical equipment unit within the horizontal plane, emitting an upward light beam from a horizontal equipment positioning light emitter on the at least one surgical equipment unit, moving the at least one surgical equipment unit within the horizontal plane until the upward light beam impinges on an elevated light marker directly above the horizontal equipment operating position of the at least one surgical equipment unit, and securing the at least one surgical equipment unit at the horizontal equipment operating position.

28. The method of claim 27 further comprising facilitating vertical positioning of the surgical patient table by placing at least one vertical patient positioning light emitter in elevated angular relationship to the horizontal patient operating position, emitting at least one angular light beam from the at least one vertical patient positioning light emitter and adjusting vertical position of the surgical patient table until the at least one angular light beam impinges on a patient reclining on the surgical patient table.

29. The method of claim 27 further comprising facilitating horizontal rotational positioning of the at least one surgical equipment unit within the at least one horizontal equipment operating position by placing at least one side light marker, placing at least one rotational equipment positioning light emitter on the at least one surgical equipment unit, emitting at least one side light beam from the at least one rotational equipment positioning light emitter and rotating the at least one surgical equipment unit until the at least one side light beam impinges on the at least one side light marker.

30. The method of claim 27 wherein emitting the upward light beam from the horizontal equipment positioning light emitter on the at least one surgical equipment unit comprises emitting the upward light beam from the horizontal equipment positioning light emitter on surgical robotic equipment, instrument holding equipment, anesthesia equipment, surgical microscopes, vitrectomy equipment or visualization equipment.

31. A surgical patient and/or equipment alignment method for optimal positioning of any combination of at least two of a surgical patient table and at least one surgical equipment unit for a surgical procedure in an operating room, the method comprising:
establishing a selected one of the at least two of the surgical patient table and the at least one surgical equipment unit as an anchor point for positioning of at least one remaining one of the at least two of the surgical patient table and the at least one surgical equipment unit by deploying the selected one of the at least two of the surgical patient table and the at least one surgical equipment unit at a corresponding selected one of a patient operating position and an equipment operating position, respectively, in the operating room; and
deploying the at least one remaining one of the at least two of the surgical patient table and the at least one surgical equipment unit relative to the selected one of the at least two of the surgical patient table and the at least one surgical equipment unit at the corresponding selected one of the patient operating position and the equipment operating position, respectively, in the operating room.

32. The method of claim 31 wherein establishing the selected one of the at least two of the surgical patient table and the at least one surgical equipment unit as the anchor point comprises establishing the surgical patient table as the anchor point.

33. The method of claim 32 wherein establishing the surgical patient table as the anchor point comprises deploying the surgical patient table in the patient operating position as the surgical patient table is guided into place by impingement of downward light beam crosshairs of a downward light beam emitted by a horizontal patient positioning light emitter into a patient on the surgical patient table.

34. The method of claim 31 wherein establishing the selected one of the at least two of the surgical patient table and the at least one surgical equipment unit as the anchor point comprises establishing the surgical equipment unit as the anchor point.

35. The method of claim 34 wherein establishing the surgical equipment unit as the anchor point comprises deploying the surgical equipment unit at the equipment operating position in the operating room by emitting at least one light beam from a horizontal equipment positioning light emitter on the surgical equipment unit onto at least one light marker.

36. The method of claim 31 further comprising deploying at least one additional surgical equipment unit relative to one of the surgical equipment unit and the surgical patient table in at least one additional equipment operating position, respectively, in the operating room.
